(19) United States
(12) Patent Application Publication
(10) Pub. No.: US 2009/0202519 A1
(43) Pub. Date: Aug. 13, 2009

(54) Compositions and methods for treating gram positive bacterial infection in a mammalian subject

(75) Inventors: Bruce Beutler, San Diego, CA (US); Phillipe Georgel, Cedex (FR)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 11/988,979

(22) PCT Filed: Jul. 20, 2006
(86) PCT No.: PCT/US06/28385

§ 371 Date: Jan. 14, 2009

Related U.S. Application Data
(63) Continuation of application No. 11/490,457, filed on Jul. 19, 2006, now abandoned.
(60) Provisional application No. 60/701,216, filed on Jul. 20, 2005.
(60) Provisional application No. 60/701,216, filed on Jul. 20, 2005.

(51) Int. Cl. A61K 39/395; A61K 31/7088; A61K 31/20; C12Q 1/68; G01N 33/569

(52) U.S. Cl. 424/130.1; 514/560; 435/6; 435/7.2

(57) ABSTRACT

Compositions and methods are provided for treating Gram positive bacterial infection in a mammalian subject. Compositions and methods are further provided for treating Gram positive bacterial skin infection in the mammalian subject. Compositions and method are provided that comprise administering to the mammalian subject an effective amount of a compound that activates Scd1 gene expression or activates Scd1 gene product.

### CROSS-REFERENCE TO RELATED APPLICATIONS

This application claims the benefit of U.S. Provisional Application No. 60/701,216, filed Jul. 20, 2005, and U.S. Application entitled “COMPOSITIONS AND METHODS FOR TREATING GRAM POSITIVE BACTERIAL INFECTION IN A MAMMALIAN SUBJECT,” filed Jul. 19, 2006, by Express Mail No. EV 670672061 US, the entire disclosures of which are incorporated herein by reference.

### STATEMENT OF GOVERNMENT SUPPORT

This invention was made by government support by Grant No. U54-AI54523 from National Institutes of Health. The Government has certain rights in this invention.

### FIELD

This invention generally relates to compositions and methods for treating Gram positive bacterial infection in a mammalian subject. The invention further relates to compositions and methods for treating Gram positive bacterial skin infection in the mammalian subject. The compositions and methods further comprise administering to the mammalian subject an effective amount of a compound that activates Scd1 gene expression or activates Scd1 gene product.

### BACKGROUND

Surface epithelia constitute the first line of defense against pathogens. This defense depends both upon barrier function and upon specific microbicidal effector molecules. For example, the mammalian skin affords physical protection partly because it is composed of tightly associated cells covered by a highly cross-linked layer of keratin, and is normally impermeable to bacteria. In humans, several genetic diseases, such as mucoepithelial dysplasia or epidemolysis bullosa, which affect the cutaneous epithelial structure at different levels, are associated with greatly increased susceptibility to infection. Vidal et al., Nat Genet. 10:229-34, 2995; Witkop et al., Am J Hum Genet 31:414-27, 1979. But the skin displays microbicidal activity even when its physical integrity is breached. It contains an arsenal of bio-active molecules, among which antimicrobial peptides (AMPs) such as defensins and cathelicidins are of critical importance to host defense against microbial invasion (reviewed in Zasloff, Nature 415:389-95, 2002; Zasloff, N Engl J Med 347:1199-200, 2002).

While AMPs are the best-studied cutaneous defense molecules, other protection systems may also exist. Monounsaturated fatty acids (MUFA), produced by the sebaceous glands, have been mentioned in this regard, and some MUFA are known to be microbicidal. Miller et al., Arch Dermatol 124:209-15, 1988; Wille and Kydonieus, Skin Pharmacol Appl Skin Physiol 16:176-87, 2003. However, their contribution to antimicrobial defense has never been established in vivo, nor is their biosynthesis known to be subject to regulation by microbial stimuli. A need exists in the art to develop improved compositions and methods that stimulate an innate immune response in response to microbial infection in mammalian subjects. A further need exists to develop improved compositions and methods for treating Gram positive bacterial infection and Gram positive bacterial skin infection in mammalian subjects.

### SUMMARY

This invention generally relates to compositions and methods for treating Gram positive bacterial infection in a mammalian subject. Compositions and methods are further provided for treating Gram positive bacterial skin infection in the mammalian subject. Compositions and methods are provided that comprise administering to the mammalian subject an effective amount of a compound that activates stearoyl CoA desaturase 1 (Scd1) gene expression or activates Scd1 gene product, stearoyl CoA desaturase.

An innate immunodeficiency phenotype in mice has been traced to a mutation affecting the structure of an enzyme essential for monounsaturated fatty acid (MUFA) synthesis. ENU-induced germline mutagenesis of C57BL/6 mice was used to isolate and identify Flake (flk), a recessive germline mutation of C57BL/6 mice. flk mutant mice are impaired in the clearance of skin infections by Streptococcus pyogenes and Staphylococcus aureus, Gram-positive pathogens that elicit innate immune responses by activating Toll-like receptor 2. Positional cloning and sequencing revealed that flk is a novel allele of the stearoyl CoA desaturase 1 gene (Scd1).

A method for treating Gram positive bacterial infection in a mammalian subject is provided comprising administering to the subject an effective amount of a compound that activates Scd1 gene expression. In one aspect, the compound is an agonist of toll-like receptor 2. In another aspect, the compound is a small chemical molecule, an antibody, an antisense nucleic acid, short hairpin RNA, or short interfering RNA. The Gram positive bacterial infection can be, for example, Streptococcus pyogenes infection or Staphlococcus aureus infection. In a further aspect, the method comprises treating the subject having a loss-of-function or reduced function mutation in the Scd1 gene.

A method for treating Gram positive bacterial infection in a mammalian subject is provided comprising administering to the subject an effective amount of a compound that activates Scd1 gene product. In one aspect, the compound is an agonist of toll-like receptor 2. In another aspect, the compound is a small chemical molecule, an antibody, an antisense nucleic acid, short hairpin RNA, or short interfering RNA. The Gram positive bacterial infection can be, for example, Streptococcus pyogenes infection or Staphlococcus aureus infection. In a further aspect, the method comprises treating the subject having a loss-of-function or reduced function mutation in the Scd1 gene.

A method for treating Gram positive bacterial infection in a mammalian subject is provided comprising administering to the subject an effective amount of a monounsaturated fatty acid. The monounsaturated fatty acid can be, for example, palmitoleate or oleate. The Gram positive bacterial infection can be, for example, Streptococcus pyogenes infection or Staphlococcus aureus infection. In one aspect, administration of the effective amount of the monounsaturated fatty acid is topical or intradermal. In another aspect, administration of the effective amount of the monounsaturated fatty acid is intramuscular, subcutaneous, intraperitoneal, or intravenous.

A method for treating Gram positive bacterial infection in a mammalian subject is provided comprising administering to the subject an effective amount of a compound that is a product of the Scd1 biosynthetic pathway. In one aspect, the compound is a monounsaturated fatty acid. The monounsaturated fatty acid can be, for example, palmitoleate or oleate. The Gram positive bacterial infection can be, for example, Streptococcus pyogenes infection or Staphlococcus aureus infection. In one aspect, administration of the effective amount of the monounsaturated fatty acid is topical or intradermal. In another aspect, administration of the effective amount of the monounsaturated fatty acid is intramuscular, subcutaneous, intraperitoneal, or intravenous.

A method for identifying a compound which modulates Gram positive bactericidal activity in cells is provided comprising: contacting the test compound with a cell-based assay system comprising a cell expressing toll-like receptor 2, providing a ligand to the assay system in an amount selected to be effective to activate toll-like receptor 2 signaling, wherein toll-like receptor 2 signaling is capable of signaling responsiveness to the ligand and modulating Scd1 gene expression, and detecting an effect of the test compound on toll-like receptor 2 signaling and on modulation of Scd1 gene expression, effectiveness of the test compound in the assay being indicative of the Gram positive bacteriocidal activity. In one aspect, the ligand is an endogenous ligand or an exogenous ligand. In a detailed aspect, the exogenous ligand is lipopolysaccharide, lipid A, di-acylated lipopeptide, tri-acylated lipopeptide, S-MALP-2, R-MALP-2, bacterial lipopeptide, Pam2CSK4, lipoteichoic acid, or zymosan A. In a further detailed aspect, the exogenous ligand is MALP-2. In a further detailed aspect, the exogenous ligand is rough lipopolysaccharide, smooth lipopolysaccharide, or lipid A from Salmonella minnesota. In a detailed aspect, the exogenous ligand is a component Gram positive bacteria, but not a component of Gram negative bacteria. In a further detailed aspect, the endogenous ligand is a lipid. The compound can be, for example, an agonist of toll-like receptor 2 pathway signaling.

In an embodiment, the method comprises the detecting step further comprising measuring activation of Scd1 gene expression or Scd1 gene product in the cell, wherein Scd1 gene expression or Scd1 gene product is activated in response to contacting the cell with the exogenous ligand.

In a further embodiment, the method is provided wherein the detecting step further comprises measuring enhanced binding of ligand to toll-like receptor 2 by the compound. The method is provided wherein the detecting step further comprises measuring increased Scd1 gene product in the cell assay. The method is provided wherein the detecting step further comprises measuring an increased Scd1 gene product activity in the cell assay. The method is provided wherein the detecting step further comprises measuring an increased monounsaturated fatty acid synthesis in the cell assay. In a further aspect, the detecting step further comprises measuring labeled ligand binding to toll-like receptor 2. The labeled ligand can be, for example, radiolabeled or fluorescent labeled.

In a further aspect, the cell assay can comprise, for example, a macrophage cell, or cells from a sebaceous gland. The cells from a sebaceous gland can be a sebocyte cell.

In an embodiment, the method further comprises providing toll-like receptor 2 to the assay system, and detecting an effect of the test compound on toll-like receptor 2 signaling in the assay system, effectiveness of the test compound in the assay being indicative of the modulation.

In an embodiment, the detecting step further comprises effecting reduced binding of ligand to toll-like receptor 2 by the compound. In a further embodiment, the detecting step further comprises effecting increased binding of ligand to toll-like receptor 2 by the compound. In a further embodiment, the detecting step further comprises measuring an increase in stearoyl CoA desaturase 1 activity in the cell assay. In a further embodiment, the detecting step further comprises measuring an increased monounsaturated fatty acid synthesis in the cell assay. In a further embodiment, the detecting step further comprises measuring an increase in Gram positive bactericidal activity in the cell assay.

A method for diagnosing a risk factor for Gram positive bacterial infection in a mammalian subject is provided comprising removing cells or tissue from the subject, contacting the cells or tissue with an endogenous ligand or exogenous ligand to toll-like receptor 2, measuring production of Scd1 gene product in the cells or tissue contacted by the ligand, and detecting reduced function or loss of function of the Scd1 gene product in the mammalian subject. The cells or tissue can be, for example, from macrophage, sebocyte, or sebaceous gland.

In one aspect, the method is provided such that the reduced function or absence of the Scd1 gene product increases risk for Gram positive bacterial infection. In another aspect, the reduced function or absence of the Scd1 gene product reduces synthesis of monounsaturated fatty acid in the cell. In a further aspect, the reduced function or absence of the Scd1 gene product reduces an inflammatory response to Gram positive bacterial infection. In a detailed aspect, the reduced function or absence of the Scd1 gene product reduces an inflammatory response at a site of injury in the patient. In a further aspect, the absence of the Scd1 gene product increases risk for conditions where inflammation is a desired defense mechanism. The ligand can be, for example, an exogenous ligand, lipotechoic acid (LTA), di-acylated lipopeptide, tri-acylated lipopeptide, S-MALP-2, bacterial lipopeptides, peptidoglycan, mannans, unmethylated CpG DNA, flagellin, or single-stranded RNA. The ligand can be, for example, an endogenous ligand, lipid, fat, sterol, lipoprotein, fatty acid, oxidized LDL, thrombospondin, or amyloid.

A method of diagnosing an Scd1 gene loss-of-function-induced disorder or a genetic predisposition therefor in a mammalian subject is provided comprising determining the presence of a mutated Scd1 protein or a nucleic acid encoding a mutated Scd1 protein in a cell sample, protein sample or nucleic acid sample obtained from the mammalian subject, wherein the presence of such a protein or nucleic acid is indicative of an Scd1 gene loss-of-function-induced disorder or a genetic predisposition therefor. In one aspect, the Scd1 gene loss-of-function-induced disorder is increased susceptibility to Gram positive bacterial infection.

In an embodiment, the method further comprises contacting the protein sample or cell sample with an anti-Scd1 antibody, and detecting the presence of a wild type or mutated Scd1 protein. In another aspect of the method the detecting step further comprises fluorescence activated cell sorting (FACS) analysis of mononuclear phagocytes or macrophages from the mammalian subject. In another aspect, the method further comprises contacting the nucleic acid sample with a labeled DNA or RNA molecule encoding a mutated Scd1 gene under hybridizing conditions and detecting the labeled DNA or RNA molecule after hybridization, wherein the detection of the labeled DNA or RNA is indicative of the presence of a nucleic acid molecule encoding a mutated Scd1 gene in the sample. In a further aspect, the method comprises contacting the nucleic acid sample with a restriction enzyme whose recognition sequence is affected by the mutation in the mutated Scd1 gene and detecting the presence or absence of fragments or the presence of altered fragments of the nucleic acid after contact with the restriction enzyme, wherein the absence of fragments or the presence of altered fragments of the nucleic acid after contact with the restriction enzyme is indicative of the presence of a nucleic acid molecule encoding a mutated Scd1 gene in the sample.

A transgenic non-human animal is provided comprising a heterologous nucleic acid, wherein the nucleic acid comprises a loss-of-function allele of a Scd1 gene, and the animal exhibits a phenotype, relative to a wild-type phenotype, comprising susceptibility to Gram positive bacterial infection. The phenotype of the transgenic non-human animal Scd1 mutant animal can be characterized, for example, by hypotrophic sebaceous gland or inability to synthesize monounsaturated fatty acids. The transgenic non-human animal can have the loss-of-function allele in the Scd1 gene, for example, an amino acid substitution at T227K. The transgenic non-human animal can be, for example, a mouse or a rat. In one aspect, a cell or cell line can be derived from the transgenic non-human animal.

An in vitro method of screening for a modulator of a Toll-like receptor 2-signaling activity is provided comprising: contacting a cell or cell line can be derived from the transgenic non-human animal with a test compound, and detecting an increase or a decrease in the amount of monounsaturated fatty acid synthesis in the cell, susceptibility to Gram positive bacterial infection, or a Toll-like receptor 2-induced macrophage activating activity, thereby identifying the test compound as a modulator of the Toll-like receptor 2-induced macrophage activating activity. An in vivo method of screening for a modulator of a Toll-like receptor 2-signaling activity is provided comprising: contacting a cell or cell line can be derived from the transgenic non-human animal with a test compound, and detecting an increase or a decrease in the amount of monounsaturated fatty acid synthesis in the cell, susceptibility to Gram positive bacterial infection, or a Toll-like receptor 2-induced macrophage activating activity, thereby identifying the test compound as a modulator of a Toll-like receptor 2-induced macrophage activating activity.

### BRIEF DESCRIPTION OF THE DRAWINGS

FIGS. 1A, 1B, 1C, and 1D show visible phenotypes observed in flake mutant mice.

FIGS. 2A, 2B, and 2C show flake mutant mice develop persistent skin infections when exposed to Gram positive bacteria.

FIGS. 3A, 3B, and 3C show mapping of the flake mutation.

FIGS. 4A and 4B show molecular characterization of the flake mutation.

FIGS. 5A and 5B show thin layer chromatography analysis of the lipid contend in wild-type and flake mutant mice.

FIGS. 6A, 6B, 6C, 6D, 6E, and 6F show palmitoleic acid has antibacterial activity in vivo.

FIGS. 7A, 7B, 7C and 7D show infection- and TLR2-dependant induction of Scd1 gene expression in mice.

FIGS. 8A, 8B, 8C and 8D show human sebocytes stimulated with MALP-2 show an inflammatory response and up-regulation of SCD1 and FADS2 genes.

FIG. 9 shows the biosynthesis of unsaturated fatty acids by the SCD1 biosynthetic pathway.

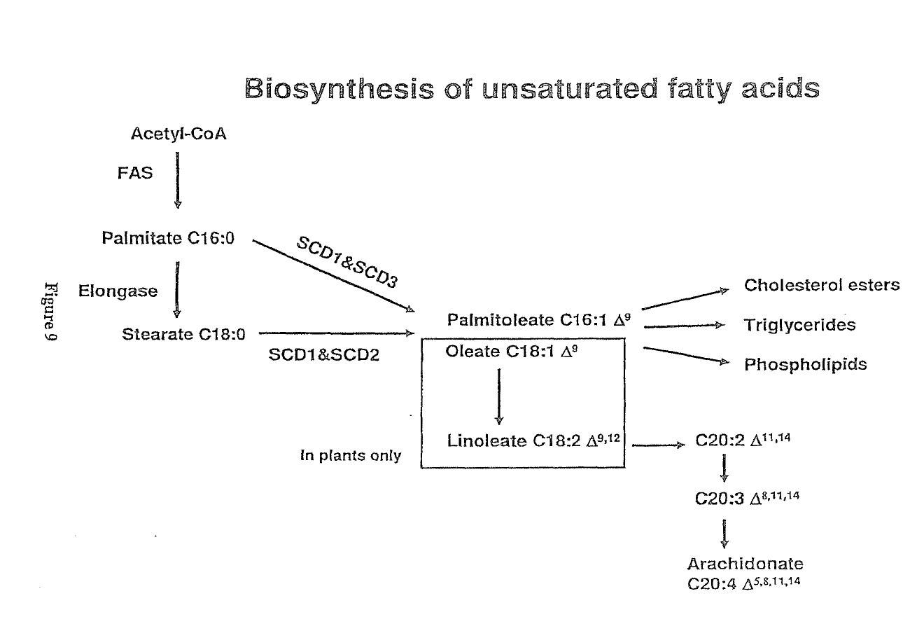

### DETAILED DESCRIPTION

This invention generally relates to compositions and methods for treating Gram positive bacterial infection in a mammalian subject. Compositions and methods are further provided for treating Gram positive bacterial skin infection in the mammalian subject. Compositions and methods are provided that comprise administering to the mammalian subject an effective amount of a compound that activates stearoyl CoA desaturase 1 (Scd1) gene expression or activates Scd1 gene product, stearoyl CoA desaturase. Methods for treating Gram positive bacterial infection in a mammalian subject are provided comprising administering to the subject an effective amount of a compound that is a monounsaturated fatty acid.

Flake (flk), an ENU-induced recessive germline mutation of C57BL/6 mice, impairs the clearance of skin infections by Streptococcus pyogenes and Staphylococcus aureus, Gram-positive pathogens that elicit innate immune responses by activating Toll-like receptor 2 (TLR2). Positional cloning and sequencing revealed that flk is a novel allele of the stearoyl CoA desaturase 1 gene (Scd1). Flake homozygotes are unable to synthesize the monounsaturated fatty acids (MUFA) palmitoleate (C16:1) and oleate (C18:1), both of which are bactericidal against Gram-positive (but not Gram-negative) organisms. Intradermal MUFA administration in S. aureus-infected mice improves bacterial clearance. In normal mice, transcription of Scd1—a gene with numerous NF-κB elements in its promoter—is strongly and specifically induced by TLR2 signaling. Similarly, the SCD1 gene is induced by TLR2 signaling in human sebocytes. These observations reveal the existence of a regulated, lipid-based antimicrobial effector pathway in mammals, and suggest new approaches to the treatment or prevention of Gram-positive bacterial infections.

“Patient”, “subject”, “vertebrate” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the antibody compositions, compounds or agents of the present invention to prevent or delay the onset of the symptoms, complications, or biochemical indicia of a disease, alleviating the symptoms or arresting or inhibiting further development of the disease, condition, or disorder (e.g., cancer, or metastatic cancer). Treatment can be prophylactic (to prevent or delay the onset of the disease, or to prevent the manifestation of clinical or subclinical symptoms thereof) or therapeutic suppression or alleviation of symptoms after the manifestation of the disease.

“Inhibitors,” “activators,” and “modulators” of Toll-like receptors in cells are used to refer to inhibitory, activating, or modulating molecules, respectively, identified using in vitro and in vivo assays for Toll-like receptors binding or signaling, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate the activity of Toll-like receptors, e.g., antagonists. Activators are agents that, e.g., bind to, stimulate, increase, open, activate, facilitate, enhance activation, sensitize or up regulate the activity of Toll-like receptors, e.g., agonists. Modulators include agents that, e.g., alter the interaction of Toll-like receptor with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring Toll-like receptor ligands, e.g., with altered activity, as well as naturally occurring and synthetic ligands, antagonists, agonists, small chemical molecules and the like. “Cell-based assays” for inhibitors and activators include, e.g., applying putative modulator compounds to a cell expressing a Toll-like receptor and then determining the functional effects on Toll-like receptor signaling, as described herein. “Cell based assays” include, but are not limited to, in vivo tissue or cell samples from a mammalian subject or in vitro cell-based assays comprising Toll-like receptor that are treated with a potential activator, inhibitor, or modulator are compared to control samples without the inhibitor, activator, or modulator to examine the extent of inhibition. Control samples (untreated with inhibitors) can be assigned a relative Toll-like receptor activity value of 100%. Inhibition of Toll-like receptor is achieved when the Toll-like receptor activity value relative to the control is about 80%, optionally 50% or 25-0%. Activation of Toll-like receptor is achieved when the Toll-like receptor activity value relative to the control is 110%, optionally 150%, optionally 200-500%, or 1000-3000% higher.

The ability of a molecule to bind to Toll-like receptor can be determined, for example, by the ability of the putative ligand to bind to Toll-like receptor immunoadhesin coated on an assay plate. Specificity of binding can be determined by comparing binding to non-Toll-like receptor.

“Test compound” refers to any compound tested as a modulator of Scd1 or toll-like receptor 2. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, test compound can be modulators that are genetically altered versions of Scd1 protein or toll-like receptor 2 protein. Typically, test compounds will be small organic molecules, peptides, lipids, or lipid analogs.

In one embodiment, antibody binding to Toll-like receptor can be assayed by either immobilizing the ligand or the receptor. For example, the assay can include immobilizing Toll-like receptor fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

“Signaling responsiveness” refers to signaling via a toll-like receptor, e.g., toll-like receptor 2. Signaling responsiveness can refer to, for example, an LPS response dependent on the membrane-spanning complex formed by Toll-like receptor 2 (TLR2) and Scd1, through which a signal is propagated. TLR2 signals, directly or indirectly, via MALP2 induction and increased Scd1 expression. The TLR2 signaling can occur, for example, in macrophages or sebaceous gland cells. Signal generating compounds for measurement in cell-based assays can be generated, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol.

“Detecting an effect of a test compound on toll-like receptor 2 signaling” can refer to a therapeutic or prophylactic effect in a mammalian subject, such as the reduction, elimination, or prevention of the disease, symptoms of the disease, or side effects of the disease in the subject. “Detecting an effect of a test compound on toll-like receptor 2 signaling” can refer to a compound having an effect in a cell-based assay, e.g., a diagnostic assay, as measured by MALP2 stimulation of TLR2 signaling and upregulation of Scd1 gene expression. A loss-of-function mutation in the Scd1 gene, e.g., a Flake mutation, impairs the clearance of skin infections by Streptococcus pyogenes and Staphylococcus aureus, Gram-positive pathogens that elicit innate immune responses by activating Toll-like receptor 2. Flake homozygotes are unable to synthesize the monounsaturated fatty acids (MUFA) palmitoleate (C16:1) and oleate (C18:1), both of which are bactericidal against Gram-positive (but not Gram-negative) organisms. Intradermal MUFA administration in S. aureus-infected mice improves bacterial clearance.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

### Antibodies as Modulators of Scd1 Gene Expression or Scd1 Gene Product or Toll-Like Receptor 2

The antibodies and antigen-binding fragments thereof described herein specifically bind to and/or activate toll-like receptor 2 (TLR2) or specifically bind to and/or activate Scd1 gene expression or Scd1 gene product. and can modulator activate an innate immune response to Gram positive bacterial infection in a mammalian subject.

Antibodies that bind TLR2 or antibodies that bind Scd1 gene product are useful as compounds that modulate signaling in cells via a toll-like receptor 2 pathway. See, for example, Takeda and Akira, Cell Microbiol 5: 143-153, 2003.

In some embodiments, the antibody or antigen-binding fragment thereof or selectively binds (e.g., competitively binds, or binds to same epitope, e.g., a conformational or a linear epitope) to an antigen that is selectively bound by an antibody produced by a hybridoma cell line. Thus, the epitope can be in close proximity spatially or functionally-associated, e.g., an overlapping or adjacent epitope in linear sequence or conformational space, to a known epitope bound by an antibody. Potential epitopes can be identified computationally using a peptide threading program, and verified using methods known in the art, e.g., by assaying binding of the antibody to mutants or fragments of the toll-like receptor 2 or Scd1 gene product, e.g., mutants or fragments of a domain of toll-like receptor 2 or Scd1 gene product.

Methods of determining the sequence of an antibody described herein are known in the art; for example, the sequence of the antibody can be determined by using known techniques to isolate and identify a cDNA encoding the antibody from the hybridoma cell line. Methods for determining the sequence of a cDNA are known in the art.

The antibodies described herein typically have at least one or two heavy chain variable regions (V_{H}), and at least one or two light chain variable regions (V_{L}). The V_{H} and V_{L} regions can be further subdivided into regions of hypervariability, termed complementarity determining regions (CDR), which are interspersed with more highly conserved framework regions (FR). These regions have been precisely defined (see, Kabat et al., Sequences of Proteins of Immunological Interest, Fifth Edition, U.S. Department of Health and Human Services, NIH Publication No. 91-3242, 1991 and Chothia et al., J. Mol. Biol. 196: 901-917, 1987). Antibodies or antibody fragments containing one or more framework regions are also useful in the invention. Such fragments have the ability to specifically bind to a domain of toll-like receptor 2 and to modulate or activate Scd1 gene product activity in a cell that has been induced by lipopolysaccharide, or to modulate or activate innate immune response to gram positive bacteria.

An antibody as described herein can include a heavy and/or light chain constant region (constant regions typically mediate binding between the antibody and host tissues or factors, including effector cells of the immune system and the first component (C1q) of the classical complement system), and can therefore form heavy and light immunoglobulin chains, respectively. For example, the antibody can be a tetramer (two heavy and two light immunoglobulin chains, which can be connected by, for example, disulfide bonds). The antibody can contain only a portion of a heavy chain constant region (e.g., one of the three domains heavy chain domains termed C_{H}1, C_{H}2, and C_{H}3, or a portion of the light chain constant region (e.g., a portion of the region termed CL).

Antigen-binding fragments are also included in the invention. Such fragments can be: (i) a F_{ab} fragment (i.e., a monovalent fragment consisting of the V_{L}, V_{H}, C_{L}, and C_{H}1 domains); (ii) a F(_{ab}′)₂ fragment (i.e., a bivalent fragment containing two F_{ab} fragments linked by a disulfide bond at the hinge region); (iii) a F_{d} fragment consisting of the V_{H} and C_{H}1 domains; (iv) a Fᵥ fragment consisting of the V_{L} and V_{H} domains of a single arm of an antibody, (v) a dAb fragment (Ward et al., Nature 341: 544-546, 1989), which consists of a V_{H} domain; and/or (vi) an isolated complementarity determining region (CDR).

Fragments of antibodies (including antigen-binding fragments as described above) can be synthesized using methods known in the art such as in an automated peptide synthesizer, or by expression of a full-length gene or of gene fragments in, for example, Scd1 gene product F(_{ab}′)₂ fragments can be produced by pepsin digestion of an antibody molecule, and F_{ab} fragments can be generated by reducing the disulfide bridges of F(_{ab}′)₂ fragments. Alternatively, F_{ab} expression libraries can be constructed (Huse et al., Science 246: 1275-81, 1989) to allow relatively rapid identification of monoclonal F_{ab} fragments with the desired specificity.

Methods of making other antibodies and antibody fragments are known in the art. For example, although the two domains of the Fv fragment, V_{L} and V_{H}, are coded for by separate genes, they can be joined, using recombinant methods or a synthetic linker that enables them to be made as a single protein chain in which the V_{L} and V_{H} regions pair to form monovalent molecules (known as single chain Fv (scFv); see e.g., Bird et al., Science 242: 423-426, 1988; Huston et al., Proc. Natl. Acad. Sci. USA 85: 5879-5883, 1988; Colcher et al., Ann. NY Acad. Sci. 880: 263-80, 1999; and Reiter, Clin. Cancer Res. 2: 245-52, 1996).

Techniques for producing single chain antibodies are also described in U.S. Pat. Nos. 4,946,778 and 4,704,692. Such single chain antibodies are encompassed within the term “antigen-binding fragment” of an antibody. These antibody fragments are obtained using conventional techniques known to those of ordinary skill in the art, and the fragments are screened for utility in the same manner that intact antibodies are screened. Moreover, a single chain antibody can form complexes or multimers and, thereby, become a multivalent antibody having specificities for different epitopes of the same target protein.

Antibodies and portions thereof that are described herein can be monoclonal antibodies, generated from monoclonal antibodies, or can be produced by synthetic methods known in the art. Antibodies can be recombinantly produced (e.g., produced by phage display or by combinatorial methods, as described in, e.g., U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 9: 1370-1372, 1991; Hay et al., Human Antibody Hybridomas 3: 81-85, 1992; Huse et al., Science 246: 1275-1281, 1989; Griffiths et al., EMBO J. 12: 725-734, 1993; Hawkins et al., J. Mol. Biol. 226: 889-896, 1992; Clackson et al., Nature 352: 624-628, 1991; Gram et al., Proc. Natl. Acad. Sci. USA 89: 3576-3580, 1992; Garrad et al., Bio/Technology 9: 1373-1377, 1991; Hoogenboom et al., Nucl. Acids Res. 19: 4133-4137, 1991; and Barbas et al., Proc. Natl. Acad. Sci. USA 88: 7978-7982, 1991).

As one example, an antibody to toll-like receptor 2 or an antibody to Scd1 gene product can be made by immunizing an animal with a TLR2 polypeptide or Scd1 polypeptide, or fragment (e.g., an antigenic peptide fragment derived from (i.e., having the sequence of a portion of) TLR24 or Scd1 gene product thereof, or a cell expressing the TLR2 antigen or Scd1 antigen or an antigenic fragment thereof. In some embodiments, antibodies or antigen-binding fragments thereof described herein can bind to a purified TLR2 or Scd1 gene product. In some embodiments, the antibodies or antigen-binding fragments thereof can bind to a TLR2 or Scd1 gene product in a tissue section, a whole cell (living, lysed, or fractionated), or a membrane fraction. Antibodies can be tested, e.g., in in vitro systems, such as measuring modulation, activation, or inhibition of Scd1 gene expression or Scd1 protein activity by MALP-2 activation of macrophages.

In the event an antigenic peptide derived from TLR2 or Scd1 gene product is used, it will typically include at least eight (e.g., 10, 15, 20, 30, 50, 100 or more) consecutive amino acid residues of a domain of TLR2 or Scd1 gene product. In some embodiments, the antigenic peptide will comprise all of the domain of TLR2 or Scd1 gene product. The antibodies generated can specifically bind to one of the proteins in their native form (thus, antibodies with linear or conformational epitopes are within the invention), in a denatured or otherwise non-native form, or both. Peptides likely to be antigenic can be identified by methods known in the art, e.g., by computer-based antigenicity-predicting algorithms. Conformational epitopes can sometimes be identified by identifying antibodies that bind to a protein in its native form, but not in a denatured form.

The host animal (e.g., a rabbit, mouse, guinea pig, or rat) can be immunized with the antigen, optionally linked to a carrier (i.e., a substance that stabilizes or otherwise improves the immunogenicity of an associated molecule), and optionally administered with an adjuvant (see, e.g.; Ausubel et al., supra). An exemplary carrier is keyhole limpet hemocyanin (KLH) and exemplary adjuvants, which will typically be selected in view of the host animal's species, include Freund's adjuvant (complete or incomplete), adjuvant mineral gels (e.g., aluminum hydroxide), surface active substances such as lysolecithin, pluronic polyols, polyanions, peptides, oil emulsions, dinitrophenol, BCG (bacille Calmette-Guerin), and Corynebacterium parvum. KLH is also sometimes referred to as an adjuvant. The antibodies generated in the host can be purified by, for example, affinity chromatography methods in which the polypeptide antigen or a fragment thereof, is immobilized on a resin.

Epitopes encompassed by an antigenic peptide will typically be located on the surface of the protein (e.g., in hydrophilic regions), or in regions that are highly antigenic (such regions can be selected, initially, by virtue of containing many charged residues). An Emini surface probability analysis of human protein sequences can be used to indicate the regions that have a particularly high probability of being localized to the surface of the protein.

The antibody can be a fully human antibody (e.g., an antibody made in a mouse or other mammal that has been genetically engineered to produce an antibody from a human immunoglobulin sequence, such as that of a human immunoglobulin gene (the kappa, lambda, alpha (IgA₁, and IgA₂), gamma (IgG₁, IgG₂, IgG₃, IgG₄), delta, epsilon and mu constant region genes or the myriad immunoglobulin variable region genes). Alternatively, the antibody can be a non-human antibody (e.g., a rodent (e.g., a mouse or rat), goat, rabbit, or non-human primate (e.g., monkey) antibody).

Human monoclonal antibodies can be generated in transgenic mice carrying the human immunoglobulin genes rather than those of the mouse. Splenocytes obtained from these mice (after immunization with an antigen of interest) can be used to produce hybridomas that secrete human mAbs with specific affinities for epitopes from a human protein (see, e.g., WO 91/00906, WO 91/10741; WO 92/03918; WO 92/03917; Lonberg et al., Nature 368: 856-859, 1994; Green et al., Nature Genet. 7: 13-21, 1994; Morrison et al., Proc. Natl. Acad. Sci. USA 81: 6851-6855, 1994; Bruggeman et al., Immunol. 7: 33-40, 1993; Tuaillon et al., Proc. Natl. Acad. Sci. USA 90: 3720-3724, 1993; and Bruggeman et al., Eur. J. Immunol. 21: 1323-1326, 1991).

The anti-TLR2 antibody or anti-Scd1 antibody can also be one in which the variable region, or a portion thereof (e.g., a CDR), is generated in a non-human organism (e.g., a rat or mouse). Thus, the invention encompasses chimeric, CDR-grafted, and humanized antibodies and antibodies that are generated in a non-human organism and then modified (in, e.g., the variable framework or constant region) to decrease antigenicity in a human. Chimeric antibodies (i.e., antibodies in which different portions are derived from different animal species (e.g., the variable region of a murine mAb and the constant region of a human immunoglobulin) can be produced by recombinant techniques known in the art. For example, a gene encoding the F_{c} constant region of a murine (or other species) monoclonal antibody molecule can be digested with restriction enzymes to remove the region encoding the murine F_{c}, and the equivalent portion of a gene encoding a human F_{c} constant region can be substituted therefore (see, e.g., European Patent Application Nos. 125,023; 184,187; 171,496; and 173,494; see also WO 86/01533; U.S. Pat. No. 4,816,567; Better et al., Science 240: 1041-1043, 1988; Liu et al., Proc. Natl. Acad. Sci. USA 84: 3439-3443, 1987; Liu et al., J. Immunol. 139: 3521-3526, 1987; Sun et al., Proc. Natl. Acad. Sci. USA 84: 214-218, 1987; Nishimura et al., Cancer Res. 47: 999-1005, 1987; Wood et al., Nature 314: 446-449, 1985; Shaw et al., J. Natl. Cancer Inst. 80: 1553-1559, 1988; Morrison et al., Proc. Natl. Acad. Sci. USA 81: 6851, 1984; Neuberger et al., Nature 312: 604, 1984; and Takeda et al., Nature 314: 452, 1984).

In a humanized or CDR-grafted antibody, at least one or two, but generally all three of the recipient CDRs (of heavy and or light immunoglobulin chains) will be replaced with a donor CDR (see, e.g., U.S. Pat. No. 5,225,539; Jones et al., Nature 321: 552-525, 1986; Verhoeyan et al., Science 239: 1534, 1988; and Beidler et al., J. Immunol. 141: 4053-4060, 1988). One need replace only the number of CDRs required for binding of the humanized antibody to toll-like receptor 2, Scd1 gene, or Scd1 gene product. The donor can be a rodent antibody, and the recipient can be a human framework or a human consensus framework. Typically, the immunoglobulin providing the CDRs is called the “donor” (and is often that of a rodent) and the immunoglobulin providing the framework is called the “acceptor.” The acceptor framework can be a naturally occurring (e.g., a human) framework, a consensus framework or sequence, or a sequence that is at least 85% (e.g., 90%, 95%, 99%) identical thereto. A “consensus sequence” is one formed from the most frequently occurring amino acids (or nucleotides) in a family of related sequences (see, e.g., Winnaker, From Genes to Clones, Verlagsgesellschaft, Weinheim, Germany, 1987). Each position in the consensus sequence is occupied by the amino acid residue that occurs most frequently at that position in the family (where two occur equally frequently, either can be included). A “consensus framework” refers to the framework region in the consensus immunoglobulin sequence. Humanized antibodies to toll-like receptor 2, Scd1 gene, or Scd1 gene product can be made in which specific amino acid residues have been substituted, deleted or added (in, e.g., in the framework region to improve antigen binding). For example, a humanized antibody will have framework residues identical to those of the donor or to amino acid a receptor other than those of the recipient framework residue. To generate such antibodies, a selected, small number of acceptor framework residues of the humanized immunoglobulin chain are replaced by the corresponding donor amino acids. The substitutions can occur adjacent to the CDR or in regions that interact with a CDR (U.S. Pat. No. 5,585,089, see especially columns 12-16). Other techniques for humanizing antibodies are described in EP 519596 A1.

An antibody to toll-like receptor 2 or an antibody to Scd1 gene product can be humanized as described above or using other methods known in the art. For example, humanized antibodies can be generated by replacing sequences of the Fv variable region that are not directly involved in antigen binding with equivalent sequences from human Fv variable regions. General methods for generating humanized antibodies are provided by Morrison, Science 229: 1202-1207, 1985; Oi et al., BioTechniques 4: 214, 1986, and Queen et al. (U.S. Pat. Nos. 5,585,089; 5,693,761, and 5,693,762). The nucleic acid sequences required by these methods can be obtained from a hybridoma producing an antibody against TLR2 or Scd1 or fragments thereof having the desired properties such as the ability to measure modulation, activation or inhibition of Scd1 gene expression or Scd1 protein activity in macrophages by MALP-2 activation. The recombinant DNA encoding the humanized antibody, or fragment thereof, can then be cloned into an appropriate expression vector.

In certain embodiments, the antibody has an effector function and can fix complement, while in others it can neither recruit effector cells nor fix complement. The antibody can also have little or no ability to bind an Fc receptor. For example, it can be an isotype or subtype, or a fragment or other mutant that cannot bind to an Fc receptor (e.g., the antibody can have a mutant (e.g., a deleted) Fc receptor binding region). Antibodies lacking the Fc region typically cannot fix complement, and thus are less likely to cause the death of the cells they bind to.

In other embodiments, the antibody can be coupled to a heterologous substance, such as a therapeutic agent (e.g., an antibiotic), or a detectable label. A detectable label can include an enzyme (e.g., horseradish peroxidase, alkaline phosphatase, .beta.-galactosidase, or acetylcholinesterase), a prosthetic group (e.g., streptavidin/biotin and avidin/biotin), or a fluorescent, luminescent, bioluminescent, or radioactive material (e.g., umbelliferone, fluorescein, fluorescein isothiocyanate, rhodamine, dichlorotriazinylamine fluorescein, dansyl chloride or phycoerythrin (which are fluorescent), luminol (which is luminescent), luciferase, luciferin, and aequorin (which are bioluminescent), and ⁹⁹mTc, ¹⁸⁸Re, ¹¹¹In, ¹²⁵I, ¹³¹I, ³⁵S or ³H (which are radioactive)).

The antibodies described herein (e.g., monoclonal antibodies) can also be used to isolate toll-like receptor 2 or Scd1 proteins or fragments thereof such as the fragment associated with modulation, activation or inhibition of Scd1 gene expression or Scd1 protein activity by MALP-2 activation of macrophages (by, for example, affinity chromatography or immunoprecipitation) or to detect them in, for example, a cell lysate or supernatant (by Western blotting, enzyme-linked immunosorbant assays (ELISAs), radioimmune assays, and the like) or a histological section. These methods permit the determination of the abundance and pattern of expression of a particular protein. This information can be useful in making a diagnosis or in evaluating the efficacy of a clinical test or treatment.

The invention also includes the nucleic acids that encode the antibodies described above and vectors and cells (e.g., mammalian cells such as CHO cells or lymphatic cells) that contain them (e.g., cells transformed with a nucleic acid that encodes an antibody that specifically binds to toll-like receptor 2 or Scd1 protein). Similarly, the invention includes cell lines (e.g., hybridomas) that make the antibodies of the invention and methods of making those cell lines.

### Immunological Detection of Scd1 Polypeptides or Toll-Like Receptor 2 Polypeptides and Modulators Thereof

In addition to the detection of Scd1 gene or toll-like receptor 2 gene and gene expression using nucleic acid hybridization technology, one can also use immunoassays to detect Scd1 or toll-like receptor 2 proteins. Such assays are useful for screening for modulators of Scd1 or toll-like receptor 2, as well as for therapeutic and diagnostic applications. Immunoassays can be used to qualitatively or quantitatively analyze Scd1 protein or toll-like receptor 2 protein. A general overview of the applicable technology can be found in Harlow & Lane, Antibodies: A Laboratory Manual, 1988.

A. Production of Antibodies

Methods of producing polyclonal and monoclonal antibodies that react specifically with Scd1 protein or toll-like receptor 2 protein are known to those of skill in the art (see, e.g., Coligan, Current Protocols in Immunology, 1991; Harlow & Lane, supra; Goding, Monoclonal Antibodies: Principles and Practice, 2d ed. 1986; and Kohler et al., Nature 256: 495-497, 1975. Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246: 1275-1281, 1989; Ward et al., Nature 341: 544-546, 1989).

A number of immunogens comprising portions of Scd1 protein or toll-like receptor 2 protein can be used to produce antibodies specifically reactive with Scd1 protein or toll-like receptor 2 protein. For example, recombinant Scd1 protein or toll-like receptor 2 protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein can also be used either in pure or impure form. The product is then injected into an animal capable of producing antibodies. Either monoclonal or polyclonal antibodies can be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see, Kohler et al., Eur. J. Immunol. 6: 511-519, 1976). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences which encode a monoclonal antibody or a binding fragment thereof by screening a DNA library from human B cells according to the general protocol outlined by Huse, et al., Science 246: 1275-1281, 1989.

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-Scd1 or toll-like receptor 2 proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular Scd1 ortholog or toll-like receptor 2 ortholog, such as human Scd1 protein or human toll-like receptor 2, can also be made, by subtracting out other cross-reacting orthologs from a species such as a non-human mammal. In this manner, antibodies that bind only to Scd1 or toll-like receptor 2 can be obtained.

Once the specific antibodies against Scd1 protein or toll-like receptor 2 protein are available, the protein can be detected by a variety of immunoassay methods. In addition, the antibody can be used therapeutically as modulators of Scd1 gene product or toll-like receptor 2. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

Scd1 protein or toll-like receptor 2 protein can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case Scd1 protein or toll-like receptor 2 protein or antigenic subsequence thereof). The antibody (e.g., anti-Scd1 gene product or anti-toll-like receptor 2) can be produced by any of a number of means well known to those of skill in the art and as described above.

Immunoassays also often use a labeling agent to specifically bind to and label the complex formed by the antibody and antigen. The labeling agent can itself be one of the moieties comprising the antibody/antigen complex. Thus, the labeling agent can be a labeled Scd1 gene product or labeled toll-like receptor 2. Alternatively, the labeling agent can be a third moiety, such a secondary antibody, that specifically binds to the antibody/Scd1 gene product or antibody/toll-like receptor 2 complex (a secondary antibody is typically specific to antibodies of the species from which the first antibody is derived). Other proteins capable of specifically binding immunoglobulin constant regions, such as protein A or protein G can also be used as the label agent. These proteins exhibit a strong non-immunogenic reactivity with immunoglobulin constant regions from a variety of species (see, e.g., Kronval et al., J. Immunol. 111: 1401-1406, 1973; Akerstrom et al., J. Immunol. 135: 2589-2542, 1985). The labeling agent can be modified with a detectable moiety, such as biotin, to which another molecule can specifically bind, such as streptavidin. A variety of detectable moieties are well known to those skilled in the art.

Throughout the assays, incubation and/or washing steps can be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-competitive assay formats: Immunoassays for detecting Scd1 gene product or toll-like receptor 2 in samples can be either competitive or noncompetitive. Noncompetitive immunoassays are assays in which the amount of antigen is directly measured. In one preferred “sandwich” assay, for example, the anti-Scd1 gene product or anti-toll-like receptor 2 antibodies can be bound directly to a solid substrate on which they are immobilized. These immobilized antibodies then capture Scd1 gene product or toll-like receptor 2 present in the test sample. Scd1 protein or toll-like receptor 2 protein thus immobilized are then bound by a labeling agent, such as a second antibody to Scd1 gene product or antibody to toll-like receptor 2 bearing a label. Alternatively, the second antibody can lack a label, but it can, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive assay formats: In competitive assays, the amount of Scd1 protein or toll-like receptor 2 protein present in the sample is measured indirectly by measuring the amount of a known, added (exogenous) Scd1 protein or toll-like receptor 2 protein displaced (competed away) from an anti-Scd1 protein or anti-toll-like receptor 2 antibody by the unknown Scd1 protein or toll-like receptor 2 protein present in a sample. In one competitive assay, a known amount of Scd1 protein or toll-like receptor 2 protein is added to a sample and the sample is then contacted with an antibody that specifically binds to Scd1 protein or toll-like receptor 2 protein. The amount of exogenous Scd1 protein or toll-like receptor 2 protein bound to the antibody is inversely proportional to the concentration of Scd1 protein or toll-like receptor 2 protein present in the sample. In a particularly preferred embodiment, the antibody is immobilized on a solid substrate. The amount of Scd1 protein or toll-like receptor 2 protein bound to the antibody can be determined either by measuring the amount of Scd1 gene product or toll-like receptor 2 present in Scd1 protein/antibody complex or toll-like receptor 2 protein/antibody complex, or alternatively by measuring the amount of remaining uncomplexed protein. The amount of Scd1 protein or toll-like receptor 2 protein can be detected by providing a labeled Scd1 protein molecule or toll-like receptor 2 molecule.

A hapten inhibition assay is another preferred competitive assay. In this assay the known Scd1 protein or toll-like receptor 2 protein is immobilized on a solid substrate. A known amount of anti-Scd1 antibody or anti-toll-like receptor 2 antibody is added to the sample, and the sample is then contacted with the immobilized Scd1 gene product or toll-like receptor 2. The amount of anti-Scd1 antibody or anti-toll-like receptor 2 antibody bound to the known immobilized Scd1 gene product or toll-like receptor 2 is inversely proportional to the amount of Scd1 protein or toll-like receptor 2 protein present in the sample. Again, the amount of immobilized antibody can be detected by detecting either the immobilized fraction of antibody or the fraction of the antibody that remains in solution. Detection can be direct where the antibody is labeled or indirect by the subsequent addition of a labeled moiety that specifically binds to the antibody as described above.

Cross-reactivity determinations: Immunoassays in the competitive binding format can also be used for crossreactivity determinations. For example, Scd1 protein or toll-like receptor 2 protein can be immobilized to a solid support. Proteins (e.g., Scd1 gene product or toll-like receptor 2 and homologs) are added to the assay that compete for binding of the antisera to the immobilized antigen. The ability of the added proteins to compete for binding of the antisera to the immobilized protein is compared to the ability of Scd1 protein or toll-like receptor 2 protein to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of Scd1 protein or toll-like receptor 2 protein, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of Scd1 protein or toll-like receptor 2 protein that is required to inhibit 50% of binding, then the second protein is said to specifically bind to the polyclonal antibodies generated to Scd1 gene product or toll-like receptor 2 immunogen.

Other assay formats: Western blot (immunoblot) analysis is used to detect and quantify the presence of Scd1 protein or toll-like receptor 2 protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind Scd1 protein or toll-like receptor 2 protein. The anti-Scd1 protein antibody or anti-toll-like receptor 2 antibody specifically bind to Scd1 gene product or toll-like receptor 2 on the solid support. These antibodies can be directly labeled or alternatively can be subsequently detected using labeled antibodies (e.g., labeled sheep anti-mouse antibodies) that specifically bind to the anti-Scd1 protein antibody or anti-toll-like receptor 2 antibody.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5: 34-41, 1986).

Reduction of non-specific binding: One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels: The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), chemiluminescent labels, and colorimetric labels such as colloidal gold or colored glass or plastic beads (e.g., polystyrene, polypropylene, latex, etc.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize Scd1 protein or toll-like receptor 2 protein, or secondary antibodies that recognize anti-Scd1 protein antibody or anti-toll-like receptor 2 antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### High Throughput Assays for Modulators of Scd1 Gene Product or TOLL-Like Receptor 2

The compounds tested as modulators of Scd1 gene product or toll-like receptor 2 can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of Scd1 protein or toll-like receptor 2 protein. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, Jan. 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Candidate compounds are useful as part of a strategy to identify drugs for treating disorders involving MALP-2 induction of macrophages via pathways involving toll-like receptor 2/Scd1 interaction. A test compound that binds to TLR2 or Scd1 is considered a candidate compound.

Screening assays for identifying candidate or test compounds that bind to TLR2 or Scd1, or modulate the activity of TLR2 or Scd1 proteins or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small chemical molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994. In some embodiments, the test compounds are activating variants of TLR2 or Scd1.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

The ability of a test compound to modulate the activity of TLR2 or Scd1 or a biologically active portion thereof can be determined, e.g., by monitoring the ability to form TLR2/Scd1 complexes in the presence of the test compound. Modulating the activity of TLR2 or Scd1 or a biologically active portion thereof can be determined by measuring MALP-2 induction of macrophages via pathways involving toll-like receptor 2/Scd1 interaction. The ability of the test compound to modulate the activity of toll-like receptor 2 or Scd1, or a biologically active portion thereof, can also be determined by monitoring the ability of the toll-like receptor 2 protein to bind to Scd1. The binding assays can be cell-based or cell-free.

The ability of a toll-like receptor 2 protein to bind to or interact with Scd1 can be determined by one of the methods described herein or known in the art for determining direct binding. In one embodiment, the ability of the toll-like receptor 2 protein to bind to or interact with Scd1 can be determined by monitoring MALP-2 induction of macrophages. Detection of the MALP-2 induction of macrophages can include detection of the expression of a recombinant Scd1 that also encodes a detectable marker such as a FLAG sequence or a luciferase. This assay can be in addition to an assay of direct binding. In general, such assays are used to determine the ability of a test compound to affect the binding of toll-like receptor 2 protein to Scd1 or activation of Scd1 protein or gene expression by toll-like receptor 2.

In general, the ability of a test compound to bind to Scd1, interfere with signaling through toll-like receptor 2, or otherwise affect MALP-2 induction of macrophages is compared to a control in which the binding or MALP-2 induction of macrophages is determined in the absence of the test compound. In some cases, a predetermined reference value is used. Such reference values can be determined relative to controls, in which case a test sample that is different from the reference would indicate that the compound binds to the molecule of interest (e.g., toll-like receptor 2) or modulates expression (e.g., modulates, activates or inhibits macrophages in a cell that has been induced by MALP-2, or modulates, activates or inhibits macrophage response to gram positive bacterial infection). A reference value can also reflect the amount of binding or MALP-2 induction of macrophages observed with a standard (e.g., the affinity of antibody for toll-like receptor 2, or modulation of Scd1 expression by MALP-2 induction). In this case, a test compound that is similar to (e.g., equal to or less than) the reference would indicate that compound is a candidate compound (e.g., binds to toll-like receptor 2 to a degree equal to or greater than a reference antibody).

This invention further pertains to novel agents identified by the above-described screening assays and uses thereof for treatments as described herein.

In one embodiment the invention provides soluble assays using Scd1 gene product or toll-like receptor 2 protein, or a cell or tissue expressing Scd1 gene product or toll-like receptor 2 protein, either naturally occurring or recombinant. In another embodiment, the invention provides solid phase based in vitro assays in a high throughput format, where Scd1 gene product or toll-like receptor 2 protein or its ligand is attached to a solid phase substrate via covalent or non-covalent interactions. Any one of the assays described herein can be adapted for high throughput screening.

In the high throughput assays of the invention, either soluble or solid state, it is possible to screen up to several thousand different modulators or ligands in a single day. This methodology can be used for Scd1 gene product or toll-like receptor 2 proteins in vitro, or for cell-based or membrane-based assays comprising Scd1 gene product or toll-like receptor 2 protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100—about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as toll-like receptors, transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I, 1993. Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Non-chemical approaches for fixing tag binders to substrates include other common methods, such as heat, cross-linking by UV radiation, and the like.

### Bispecific Compounds as Modulators of Scd1 and Toll-Like Receptor 2

In one aspect, a method for identifying candidate or test bispecific compounds is provided which reduce the concentration of an agent in the serum and/or circulation of a non-human animal. Compounds selected or optimized using the instant methods can be used to treat subjects that would benefit from administration of such a compound, e.g., human subjects.

Candidate compounds that can be tested in an embodiment of the methods of the present invention are bispecific compounds. As used herein, the term “bispecific compound” includes compounds having two different binding specificities. Exemplary bispecific compounds include, e.g., bispecific antibodies, heteropolymers, and antigen-based heteropolymers.

Bispecific molecules that can be tested in an embodiment of the invention preferably include a binding moiety that is specific for Scd1, preferably human Scd1, crosslinked to a second binding moiety specific for a targeted agent (e.g. a distinct antibody or an antigen). Examples of binding moieties specific for toll-like receptor 2 include, but are not limited to, toll-like receptor 2 ligands, e.g. MALP-2 or, in preferred embodiments, antibodies to toll-like receptor 2.

In another embodiment, novel toll-like receptor 2 binding molecules can be identified based on their ability to bind to toll-like receptor 2. For example, libraries of compounds or small chemical molecules can be tested cell-free binding assay. Any number of test compounds, e.g., peptidomimetics, small chemical molecules or other drugs can be used for testing and can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small chemical molecule libraries of compounds (Lam, Anticancer Drug Des. 12: 145, 1997).

In many drug screening programs which test libraries of modulating agents and natural extracts, high throughput assays are desirable in order to maximize the number of modulating agents surveyed in a given period of time. Assays which are performed in cell-free systems, such as can be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as can be manifest in an alteration of binding affinity with upstream or downstream elements.

In another embodiment, phage display techniques known in the art can be used to identify novel TLR2 or Scd1 binding molecules.

In one embodiment, the invention provides assays for screening candidate or test compounds which bind to TLR2 or Scd1 or biologically active portion thereof.

Cell-based assays for identifying molecules that bind to TLR2 or Scd1 can be used to identify additional agents for use in bispecific compounds of the invention. For example, cells expressing TLR2 or Scd1 can be used in a screening assay. For example, compounds which produce a statistically significant change in binding to TLR2 or Scd1 can be identified.

In one embodiment, the assay is a cell-free assay in which a toll-like receptor 2 binding molecule is identified based on its ability to bind to TLR2 or Scd1 protein in vitro. The TLR2 or Scd1 protein binding molecule can be provided and the ability of the protein to bind TLR2 or Scd1 protein can be tested using art recognized methods for determining direct binding. Determining the ability of the protein to bind to a target molecule can be accomplished, e.g., using a technology such as real-time Biomolecular Interaction Analysis (BIA). Sjolander et al., Anal. Chem. 63: 2338-2345, 1991, and Szabo et al., Curr. Opin. Struct. Biol. 5: 699-705, 1995. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

The cell-free assays of the present invention are amenable to use of both soluble and/or membrane-bound forms of proteins. In the case of cell-free assays in which a membrane-bound form a protein is used it can be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Suitable assays are known in the art that allow for the detection of protein-protein interactions (e.g., immunoprecipitations, two-hybrid assays and the like). By performing such assays in the presence and absence of test compounds, these assays can be used to identify compounds that modulate (e.g., inhibit or enhance) the interaction of a protein of the invention with a target molecule(s).

Determining the ability of the protein to bind to or interact with a target molecule can be accomplished, e.g., by direct binding. In a direct binding assay, the protein could be coupled with a radioisotope or enzymatic label such that binding of the protein to a target molecule can be determined by detecting the labeled protein in a complex. For example, proteins can be labeled with ¹²⁵I, ³⁵S, ¹⁴C, or ³H, either directly or indirectly, and the radioisotope detected by direct counting of radioemmission or by scintillation counting. Alternatively, molecules can be enzymatically labeled with, for example, horseradish peroxidase, alkaline phosphatase, or luciferase, and the enzymatic label detected by determination of conversion of an appropriate substrate to product.

Typically, it will be desirable to immobilize either a protein of the invention or its binding protein to facilitate separation of complexes from uncomplexed forms of one or both of the proteins, as well as to accommodate automation of the assay. Binding to an upstream or downstream binding element, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/TLR2 (GST/TLR2) fusion proteins can be adsorbed onto glutathione sepharose beads (Sigma Chemical, St. Louis, Mo.) or glutathione derivatized microtitre plates, which are then combined with the cell lysates, e.g. ³⁵S-labeled, and the test modulating agent, and the mixture incubated under conditions conducive to complex formation, e.g., at physiological conditions for salt and pH, though slightly more stringent conditions can be used. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of TLR2-binding protein found in the bead fraction quantitated from the gel using standard electrophoretic techniques.

Other techniques for immobilizing proteins on matrices are also available for use in the subject assay. For instance, biotinylated molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical).

It is also within the scope of this invention to determine the ability of a compound to modulate the interaction between TLR2 and Scd1, without the labeling of any of the interactants. For example, a microphysiometer can be used to detect the interaction of a protein of the invention with its target molecule without the labeling of either the protein or the target molecule. McConnell et al., Science 257: 1906-1912, 1992. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

Antigen-based heteropolymers that can be tested in the present invention preferentially include a binding moiety that is specific for TLR2 or Scd1, preferably human TLR2 or Scd1, crosslinked to an antigen that is recognized by an autoantibody. Examples of antigens recognized by autoantibodies include, but are not limited to, any one of the following: factor VIII (antibodies associated with treatment of hemophilia by replacement recombinant factor VIII); the muscle acetylcholine receptor (the antibodies are associated with the disease myasthenia gravis); cardiolipin (associated with the disease lupus); platelet associated proteins (associated with the disease idiopathic thrombocytopenic purpura); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is well-characterized and is associated with disease pathogenesis.

Exemplary heteropolymers and antigen-based heteropolymers for testing in the instant invention and methods of making them are known in the art. For example, exemplary heteropolymers are taught in WO 03007971A1; U.S. 20020103343A1; U.S. Pat. No. 5,879,679; U.S. Pat. No. 5,487,890; U.S. Pat. No. 5,470,570; WO 9522977A1; WO/02075275A3, WO/0246208A2 or A3, WO/0180883A1, WO/0145669A1, WO 9205801A1, Lindorfer et al., J. Immunol. Methods. 248: 125, 2001; Hahn et al., J. Immunol. 166: 1057, 2001; Nardin et al., J. Immunol. Methods. 211: 21, 1998; Kuhn et al., J. Immunol. 160: 5088, 1998; Taylor et al., Cancer Immunol. Immunother. 45: 152, 1997; Taylor et al., J. Immunol. 159: 4035, 1997; and Taylor et al., J. Immunol. 148: 2462, 1992. In addition, variant forms of these heteropolymers can be made. For example, in one embodiment, forms of bispecific molecules made using different linking chemistries can be used. Exemplary reagents that can be used to cross-link the components of a bispecific molecule include: polyethelyene glycol, SATA, SMCC, as well others known in the art, and available, e.g., from Pierce Biotechnology. Exemplary forms of bispecific molecules that can be tested are described in U.S. Ser. No. 60/411,731, filed on Sep. 16, 2002, the contents of which are incorporated herein by reference.

In another embodiment, different multimeric forms of bispecific molecules can be made (e.g., dimer, trimer, tetramer, pentamer, or higher multimer forms). In another embodiment, purified forms of bispecific molecules can be tested, e.g., as described in U.S. Ser. No. 60/380,211, filed on May 13, 2002, the contents of which are incorporated herein by reference.

In another embodiment, when one of the binding moieties of the heteropolymer is an antibody, antibodies of different isotypes (e.g., IgA, IgD, IgE, IgG1, IgG₂ (e.g., IgG₂a), IgG₃, IgG₄, or IgM) can be used. In another embodiment, portions of an antibody molecule (e.g., Fab fragments) can be used for one of the binding moieties. In a preferred embodiment at least one of the binding moieties is an antibody comprising an Fc domain. In one embodiment, the antibody is a mouse antibody.

In another embodiment, the effect of modifications to antibodies can be tested, e.g., the effect of deimmunization of the antibody, e.g., as described in U.S. Ser. No. 60/458,869, filed on Mar. 28, 2003 can be tested.

In methods provided in the present invention, the concentration of an agent, e.g. pathogenic agent, in the serum, circulation and/or tissue of the non-human animal can be reduced by at least e.g. about 20%, about 30%, about 40%, about 50%, about 60%, about 70%, about 80%, about 90% or about 100%.

In another embodiment, the concentration of an agent in the serum, circulation and/or tissue of a subject can be measured indirectly. For example, pathology resulting from the presence of the agent in the serum and/or circulation can be measured, e.g., by examining tissue samples from the animal. Another indirect measurement of the concentration of an agent in the serum, circulation and/or tissue of the non-human animal is measurement of the ability of the agent to cause infection in the non-human animal. For example, the effect of the bispecific compound on clinical signs and symptoms of infection can be measured. The ability of the bispecific compound to inhibit the spread of infection, e.g., from one organ system to another or from one individual to another can also be tested.

In another embodiment the ability of the bispecific compound to bind to cells bearing TLR2 or Scd1 in the non-human animal is measured. For example, in one embodiment, determining the ability of the bispecific compound to bind to a TLR2 or Scd1 target molecule can also be accomplished using a technology such as real-time Biomolecular Interaction Analysis (BIA) (Sjolander et al., Anal. Chem. 63: 2338-2345, 1991 and Szabo et al., Curr. Opin. Struct. Biol. 5: 699-705, 1995). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, the destruction of the agent by cells in the non-human animal (e.g., killing by macrophage) is measured.

Compounds that reduce the concentration of the agent in the serum and/or circulation of the non-human animal (as compared with concentrations observed in non-human animals that do not receive the bispecific compound) can be selected.

Compounds for testing in the subject assays can be selected from among a plurality of compounds tested. In another embodiment, bispecific compounds for testing in the instant assays may have already been identified as being capable of binding TLR2 or Scd1, e.g., in an in vitro assay and can be further evaluated or optimized using the instant assays. In such cases, the ability of a bispecific compound to reduce the concentration of an agent in the serum and/or circulation can be compared to another bispecific compound or a non-optimized version of the same compound to determine its ability reduce the concentration of the agent in the serum and/or circulation.

In preferred embodiments, the bispecific compounds of the instant invention are administered at concentrations in the range of approximately 1 μg compound/kg of body weight to approximately 100 μg compound/kg of body weight. As defined herein, a therapeutically effective amount of a bispecific compound (i.e., an effective dosage) ranges from about 0.01 to 5000 μg/kg body weight, preferably about 0.1 to 500 μg/kg body weight, more preferably about 2 to 80 μg/kg body weight, and even more preferably about 5 to 70 μg/kg, 10 to 60 μg/kg, 20 to 50 μg/kg, 24 to 41 μg/kg, 25 to 40 μg/kg, 26 to 39 μg/kg, 27 to 38 μg/kg, 28 to 37 μg/kg, 29 to 36 μg/kg, 30 to 35 μg/kg, 31 to 34 μg/kg or 32 to 33 μg/kg body weight. The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, the animal is treated with bispecific compound in the range of between about 1 to 500 μg/kg body weight following intravenous (iv) injection of an agent. It will also be appreciated that the effective dosage of a bispecific compound used for treatment can increase or decrease over the course of a particular treatment. Changes in dosage may result and become apparent from the results of diagnostic assays as described herein.

The route of administration of test compounds and/or agents can be intravenous (iv) injection into the circulation of the animal. Other administration routes include, but are not limited to, topical, parenteral, subcutaneous, or by inhalation. The term “parenteral” includes injection, e.g. by subcutaneous, intravenous, or intramuscular routes, also including localized administration, e.g., at a site of disease or injury. Sustained release of compounds from implants is also known in the art. One skilled in the pertinent art will recognize that suitable dosages will vary, depending upon such factors as the nature of the disorder to be treated, the patient's body weight, age, and general condition, and the route of administration. Preliminary doses can be determined according to animal tests, and the scaling of dosages for human administration are performed according to art-accepted practices.

The candidate compounds and agents can be administered over a range of doses to the animal. When the agent is also administered to the animal, the candidate compound can be administered either before, at the same time, or after, administration of the agent.

TLR2- or Scd1-expressing transgenic animals, e.g. mice, of the present invention can be used to screen or evaluate candidate compounds useful for treating disorders or diseases in humans that are associated with the presence of unwanted agents in the serum and/or circulation of a subject, such as autoantibodies, infectious agents, or toxins.

Exemplary targeted agents that can be bound by the bispecific compounds of the present invention include blood-borne agents, including, but not limited to, any of the following: viruses, viral particles, toxins, bacteria, polynucleotides, antibodies, e.g., autoantibodies associated with an autoimmune disorder. In one embodiment, exemplary targeted viral agents include, but are not limited to, any one of the following: cytomegalovirus, influenza, Newcastle disease virus, vesicular stomatitis virus, rabies virus, herpes simplex virus, hepatitis, adenovirus-2, bovine viral diarrhea virus, human immunodeficiency virus (HIV), dengue virus, Marburg virus, Epstein-Barr virus.

Exemplary Gram-positive bacterial targets Streptococcus pyogenes, Staphylococcus aureus, Mycobacterium tuberculosis, Streptococcus pneumoniae, or Bacillus subtilis. Any of the methods and compositions described above are useful for the treatment of skin infections, community-acquired pneumonia, upper and lower respiratory tract infections, skin and soft tissue infections, hospital-acquired lung infections, bone and joint infections, respiratory tract infections, acute bacterial otitis media, bacterial pneumonia, urinary tract infections, complicated infections, noncomplicated infections, pyelonephritis, intra-abdominal infections, deep-seated abcesses, bacterial sepsis, central nervous system infections, bacteremia, wound infections, peritonitis, meningitis, infections after burn, urogenital tract infections, gastro-intestinal tract infections, pelvic inflammatory disease, endocarditis, and other intravascular infections. The infections to be treated may be caused by Gram-positive bacteria. These include, without limitation, infections by, Staphylococcus aureus, Staphylococcus epidermidis, Enterococcus faecalis, Enterococcus faecium, Clostridium perfringens, Clostridium difficile, Streptococcus pyogenes, Streptococcus pneumoniae, other Streptococcus spp., and other Clostridium spp. More specifically, the infections may be caused by a Gram-positive coccus, or by a drug-resistant Gram-positive coccus. Exemplary Gram-positive cocci are, without limitation, S. aureus, S. epidermidis, S. pneumoniae, S. pyogenes, M. catarrhalis, C. difficile, H. pylori, Chlamydia spp., and Enterococcus spp.

Bacteremia can be caused by gram-negative or gram-positive bacteria. Gram-negative bacteria have thin walled cell membranes consisting of a single layer of peptidoglycan and an outer layer of lipopolysaccharide, lipoprotein, and phospholipid. Exemplary gram-negative organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species. Gram-positive bacteria have a thick cell membrane consisting of multiple layers of peptidoglycan and an outside layer of teichoic acid. Exemplary gram-positive organisms include, but are not limited to, Staphylococcus aureus, coagulase-negative staphylococci, streptococci, enterococci, corynebacteria, and Bacillus species.

In one embodiment, the targeted agent is resistant to traditional therapies, e.g., is resistant to antibiotics.

In one embodiment, in performing an assay of the invention, the agent is administered to the transgenic animal, e.g., prior to, simultaneously with, or after administration of a bispecific compound.

The bispecific compounds of the present invention, or any portion thereof, can be modified to enhance their half life. Peptide analogs are commonly used in the pharmaceutical industry as non-peptide drugs with properties analogous to those of the template peptide. These types of non-peptide compounds are termed “peptide mimetics” or “peptidomimetics” (Fauchere, Adv. Drug Res. 15: 29, 1986; Veber et al., TINS p. 392, 1985; and Evans et al., J. Med. Chem. 30: 1229, 1987, which are incorporated herein by reference) and are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), such as an antigen polypeptide, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins Weinstein, B., ed., Marcel Dekker, New York, p. 267, 1983; Spatola, A. F., Vega Data, Vol. 1, Issue 3, “Peptide Backbone Modifications,” 1983; Morley, Trends. Pharm. Sci. pp. 463-468, 1980; Hudson et al., Int. J. Pept. Prot. Res. 14: 177-185, 1979 (—CH₂NH—, CH₂CH₂—); Spatola et al., Life. Sci. 38: 1243-1249, 1986 (—CH₂—S); Hann, J. Chem. Soc. Perkin. Trans. 1: 307-314, 1982 (—CH—CH—, cis and trans); Almquist et al., J. Med. Chem. 23: 1392-1398, 1980 (—COCH₂—); Jennings-White et al., Tetrahedron Lett. 23: 2533, 1982 (—COCH₂—); Szelke et al., European Patent Application No. EP 45665 CA: 97: 39405, 1982 (—CH(OH)CH₂—); Holladay et al., Tetrahedron. Lett. 24: 4401-4404, 1983 (—C(OH)CH₂—); and Hruby, Life Sci. 31: 189-199, 1982 (—CH₂—S—); each of which is incorporated herein by reference. A particularly preferred non-peptide linkage is —CH₂NH—. Such peptide mimetics can have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) can be used to generate more stable peptides. In addition, constrained peptides can be generated by methods known in the art (Rizo et al., Annu. Rev. Biochem. 61: 387, 1992, incorporated herein by reference); for example, by adding internal cysteine residues capable of forming intramolecular disulfide bridges which cyclize the peptide.

Such modified polypeptides can be produced in prokaryotic or eukaryotic host cells. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual, 2nd Ed., Cold Spring Harbor, N.Y., 1989; Berger et al., Methods in Enzymology, Volume 152, Guide to Molecular Cloning Techniques, 1987, Academic Press, Inc., San Diego, Calif.; Merrifield, J. Am. Chem. Soc. 91: 501, 1969; Chaiken, CRC Crit. Rev. Biochem. 11: 255, 1981; Kaiser et al., Science 243: 187, 1989; Merrifield, Science 232: 342, 1986; Kent, Annu. Rev. Biochem. 57: 957, 1988; and Offord, Semisynthetic Proteins, Wiley Publishing, 1980, which are incorporated herein by reference).

Polypeptides can be produced, typically by direct chemical synthesis, and used as a binding moiety of a heteropolymer. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal modifications such as amidation, as well as other terminal modifications, including cyclization, can be incorporated into various embodiments of the test compounds. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

### Construction of Transgenic Animals

In one aspect, the present invention provides a animal whose genome contains a polynucleotide encoding TLR2 or Scd1 operably linked to a promoter such that the non-human or human TLR2 gene or Scd1 gene is functionally expressed in the macrophages of the animal, or the non-human or human TLR2 or Scd1 is a gain of function mutation in the macrophage of the animal. The present invention further provides methods for making a transgenic non-human animal expressing non-human or human TLR2 or Scd1 in the macrophages of the animal.

The transgenic animal used in the methods of the invention can be, e.g., a mammal, a bird, a reptile or an amphibian. Suitable mammals for uses described herein include: rodents; ruminants; ungulates; domesticated mammals; and dairy animals. Preferred animals include: rodents, goats, sheep, camels, cows, pigs, horses, oxen, llamas, chickens, geese, and turkeys. In a preferred embodiment, the non-human animal is a mouse.

Various methods of making transgenic animals are known in the art (see, e.g., Watson, et al., “The Introduction of Foreign Genes Into Mice,” in Recombinant DNA, 2d Ed., W. H. Freeman & Co., New York, pp. 255-272, 1992; Gordon, Intl. Rev. Cytol. 115: 171-229, 1989; Jaenisch, Science 240: 1468-1474, 1989; Rossant, Neuron 2: 323-334, 1990). An exemplary protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. An exemplary protocol for the production of a transgenic rat can be found in Bader et al., Clinical and Experimental Pharmacology and Physiology, Supp. 3: S81-S87, 1996. An exemplary protocol for the production of a transgenic cow can be found in Transgenic Animal Technology, A Handbook, 1994, ed., Carl A. Pinkert, Academic Press, Inc. An exemplary protocol for the production of a transgenic sheep can be found in Transgenic Animal Technology, A Handbook, 1994, ed., Carl A. Pinkert, Academic Press, Inc. Several exemplary methods are set forth in more detail below.

A. Injection into the Pronucleus

Transgenic animals can be produced by introducing a nucleic acid construct according to the present invention into egg cells. The resulting egg cells are implanted into the uterus of a female for normal fetal development, and animals which develop and which carry the transgene are then backcrossed to create heterozygotes for the transgene. Embryonal target cells at various developmental stages are used to introduce the transgenes of the invention. Different methods are used depending on the stage of development of the embryonal target cell(s). Exemplary methods for introducing transgenes include, but are not limited to, microinjection of fertilized ovum or zygotes (Brinster et al., Proc. Natl. Acad. Sci. USA 82: 4438-4442, 1985), and viral integration (Jaenisch, Proc. Natl. Acad. Sci. USA 73: 1260-1264, 1976; Jahner et al., Proc. Natl. Acad. Sci. USA 82: 6927-6931, 1985; Van der Putten et al., Proc. Natl. Acad. Sci. USA 82: 6148-6152, 1985). Procedures for embryo manipulation and microinjection are described in, for example, Manipulating the Mouse Embryo (Cold Spring Harbor Laboratory Press, Cold Spring Harbor, N.Y., 1986, the contents of which are incorporated herein by reference). Similar methods are used for production of other transgenic animals.

In an exemplary embodiment, production of transgenic mice employs the following steps. Male and female mice, from a defined inbred genetic background, are mated. The mated female mice are previously treated with pregnant mare serum, PMS, to induce follicular growth and human chorionic gonadotropin, hCG, to induce ovulation. Following mating, the female is sacrificed and the fertilized eggs are removed from her uterine tubes. At this time, the pronuclei have not yet fused and it is possible to visualize them using light microscopy. In an alternative protocol, embryos can be harvested at varying developmental stages, e.g. blastocysts can be harvested. Embryos are recovered in a Dulbecco's modified phosphate buffered saline (DPBS) and maintained in Dulbecco's modified essential medium (DMEM) supplemented with 10% fetal bovine serum.

Foreign DNA or the recombinant construct (e.g. TLR2 or Scd1 expression construct) is then microinjected (100-1000 molecules per egg) into a pronucleus. Microinjection of an expression construct can be performed using standard micro manipulators attached to a microscope. For instance, embryos are typically held in 100 microliter drops of DPBS under oil while being microinjected. DNA solution is microinjected into the male pronucleus. Successful injection is monitored by swelling of the pronucleus. Shortly thereafter, fusion of the pronuclei (a female pronucleus and a male pronucleus) occurs and, in some cases, foreign DNA inserts into (usually) one chromosome of the fertilized egg or zygote. Recombinant ES cells, which are prepared as set forth below, can be injected into blastocysts using similar techniques.

B. Embryonic Stem Cells

In another method of making transgenic mice, recombinant DNA molecules of the invention can be introduced into mouse embryonic stem (ES) cells. Resulting recombinant ES cells are then microinjected into mouse blastocysts using techniques similar to those set forth in the previous subsection.

ES cells are obtained from pre-implantation embryos and cultured in vitro (Evans et al., Nature 292: 154-156, 1981; Bradley et al., Nature 309: 255-258, 1984; Gossler et al., Proc. Natl. Acad. Sci. USA 83: 9065-9069, 1986; Robertson et al., Nature 322: 445-448, 1986). Any ES cell line that is capable of integrating into and becoming part of the germ line of a developing embryo, so as to create germ line transmission of the targeting construct, is suitable for use herein. For example, a mouse strain that can be used for production of ES cells is the 129J strain. A preferred ES cell line is murine cell line D3 (American Type Culture Collection catalog no. CRL 1934). The ES cells can be cultured and prepared for DNA insertion using methods known in the art and described in Robertson, Teratocarcinomas and Embryonic Stem Cells: A Practical Approach, E. J. Robertson, ed. IRL Press, Washington, D.C., 1987, in Bradley et al., Current Topics in Devel. Biol. 20: 357-371, 1986 and in Hogan et al., Manipulating the Mouse Embryo: A Laboratory Manual, Cold Spring Harbor Laboratory Press, Cold Spring Harbor, N.Y., 1986, the contents of which are incorporated herein by reference.

The expression construct can be introduced into the ES cells by methods known in the art, e.g., those described in Sambrook et al., Molecular Cloning: A Laboratory Manual, 2nd Ed., ed., Cold Spring Harbor laboratory Press: 1989, the contents of which are incorporated herein by reference. Suitable methods include, but are not limited to, electroporation, microinjection, and calcium phosphate treatment methods. The expression construct (e.g. TLR2 or Scd1) to be introduced into the ES cell is preferably linear. Linearization can be accomplished by digesting the DNA with a suitable restriction endonuclease selected to cut only within the vector sequence and not within the gene (e.g. TLR2 or Scd1 gene).

After introduction of the expression construct, the ES cells are screened for the presence of the construct. The cells can be screened using a variety of methods. Where a marker gene is employed in the construct, the cells of the animal can be tested for the presence of the marker gene. For example, where the marker gene is an antibiotic resistance gene, the cells can be cultured in the presence of an otherwise lethal concentration of antibiotic (e.g. G418 to select for neo). Those cells that survive have presumably integrated the transgene construct. If the marker gene is a gene that encodes an enzyme whose activity can be detected (e.g., .beta.-galactosidase), the enzyme substrate can be added to the cells under suitable conditions, and the enzymatic activity can be analyzed. Alternatively, or additionally, ES cell genomic DNA can be examined directly. For example, the DNA can be extracted from the ES cells using standard methods and the DNA can then be probed on a Southern blot with a probe or probes designed to hybridize specifically to the transgene. The genomic DNA can also be amplified by PCR with probes specifically designed to amplify DNA fragments, of a particular size and sequence of the transgene such that, only those cells containing the targeting construct will generate DNA fragments of the proper size.

C. Implantation

The zygote harboring a recombinant nucleic acid molecule of the invention (e.g. TLR2 or Scd1) is implanted into a pseudo-pregnant female mouse that was obtained by previous mating with a vasectomized male. In a general protocol, recipient females are anesthetized, paralumbar incisions are made to expose the oviducts, and the embryos are transformed into the ampullary region of the oviducts. The body wall is sutured and the skin closed with wound clips. The embryo develops for the full gestation period, and the surrogate mother delivers the potentially transgenic mice. Finally, the newborn mice are tested for the presence of the foreign or recombinant DNA. Of the eggs injected, on average 10% develop properly and produce mice. Of the mice born, on average one in four (25%) are transgenic for an overall efficiency of 2.5%. Once these mice are bred they transmit the foreign gene in a normal (Mendelian) fashion linked to a mouse chromosome.

D. Screening for the Presence of the Transgenic Construct

Transgenic animals can be identified after birth by standard protocols. DNA from tail tissue can be screened for the presence of the transgene construct, e.g., using southern blots and/or PCR. Offspring that appear to be mosaics are then crossed to each other if they are believed to carry the transgene in order to generate homozygous animals. If it is unclear whether the offspring will have germ line transmission, they can be crossed with a parental or other strain and the offspring screened for heterozygosity. The heterozygotes are identified by southern blots and/or PCR amplification of the DNA. The heterozygotes can then be crossed with each other to generate homozygous transgenic offspring. Homozygotes can be identified by Southern blotting of equivalent amounts of genomic DNA from mice that are the product of this cross, as well as mice that are known heterozygotes and wild type mice. Probes to screen the southern blots can be designed based on the sequence of the human or non-human TLR2 or Scd1 gene, or the marker gene, or both.

Other means of identifying and characterizing the transgenic offspring are known in the art. For example, western blots can be used to assess the level of expression of the gene introduced in various tissues of these offspring by probing the western blot with an antibody against the protein encoded by the gene introduced (e.g., the human or non-human TLR2 or Scd1 protein), or an antibody against the marker gene product, where this gene is expressed.

In situ analysis, such as fixing the cells and labeling with an antibody, and/or FACS (fluorescence activated cell sorting) analysis of various cells, e.g. erythrocytes, from the offspring can be performed using suitable antibodies to look for the presence or absence of the transgene product. For example, to verify expression of TLR2 or Scd1 in macrophages, flow cytometry can be performed using antibodies specific for human TLR2 or Scd1, that are directly conjugated or used in conjunction with a secondary antibody that is fluorophore-conjugated and recognizes the antibody for TLR2 or Scd1. In this analysis, human erythrocytes can be used as a positive control and normal mouse erythrocytes can be used as a negative control for the presence of TLR2 or Scd1.

E. Mice Containing Multiple Transgenes or an Additional Mutation

Transgenic mice expressing TLR2 or Scd1 as described herein can be crossed with mice that a) harbor additional transgene(s), or b) contain mutations in other genes. Mice that are heterozygous or homozygous for each of the mutations can be generated and maintained using standard crossbreeding procedures. Examples of mice that can be bred with mice containing a TLR2 or Scd1 transgene include, but are not limited to, mouse strains which are more prone to an auto-immune disease, such as mouse strains which are models for Lupus, e.g. mouse strains NZB/W, MRL+ or SJL.

The invention further pertains to cells derived from transgenic animals. Because certain modifications can occur in succeeding generations due to either mutation or environmental influences, such progeny may not, in fact, be identical to the parent cell, but are still included within the scope of the term as used herein.

### Recombinant Nucleic Acid Techniques

The nucleic acids used to practice this invention, whether RNA, iRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams, J. Am. Chem. Soc. 105: 661, 1983; Belousov, Nucleic Acids Res. 25: 3440-3444, 1997; Frenkel, Free Radic. Biol. Med. 19: 373-380, 1995; Blommers, Biochemistry 33: 7886-7896, 1994; Narang, Meth. Enzymol. 68: 90, 1979; Brown Meth. Enzymol. 68: 109, 1979; Beaucage, Tetra. Lett. 22: 1859, 1981; U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which can be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y., 1993.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, adioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat. Genet. 15: 333-335, 1997; yeast artificial chromosomes (YAC); bacterial artificial chromosomes (BAC); P1 artificial chromosomes, see, e.g., Woon, Genomics 50: 306-316, 1998; P1-derived vectors (PACs), see, e.g., Kern, Biotechniques 23:120-124, 1997; cosmids, recombinant viruses, phages or plasmids.

The invention provides fusion proteins and nucleic acids encoding them. A Scd1 gene product or toll-like receptor 2 polypeptide can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif 12: 404-414, 1998). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell. Biol. 12: 441-53, 1993.

A. Transcriptional Control Elements

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

B. Expression Vectors and Cloning Vehicles

The invention provides expression vectors and cloning vehicles comprising nucleic acids of the invention, e.g., sequences encoding the proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature 328: 731, 1987; Schneider, Protein Expr. Purif. 6435: 10, 1995; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picomnaviridiae. Chimeric vectors can also be employed which exploit advantageous merits of each of the parent vector properties (See e.g., Feng, Nature Biotechnology 15: 866-870, 1997). Such viral genomes can be modified by recombinant DNA techniques to include the nucleic acids of the invention; and can be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher, J. Virol. 66: 2731-2739, 1992; Johann, J. Virol. 66: 1635-1640, 1992). Adeno-associated virus (AAV)-based vectors can be used to adioimmun cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther. 3: 957-964, 1996.

“Expression cassette” as used herein refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination. Thus, expression cassettes also include plasmids, expression vectors, recombinant viruses, any form of recombinant “naked DNA” vector, and the like.

“Vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

C. Host Cells and Transformed Cells

The invention also provides a transformed cell comprising a nucleic acid sequence of the invention, e.g., a sequence encoding a polypeptide of the invention, or a vector of the invention. The host cell can be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

D. Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the Scd1 protein or toll-like receptor 2 sequences, or subsequences thereof.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4: 560, 1989; Landegren, Science 241: 1077, 1988; Barringer, Gene 89: 117, 1990); transcription amplification (see, e.g., Kwoh, Proc. Natl. Acad. Sci. USA 86: 1173, 1989); and, self-sustained sequence replication (see, e.g., Guatelli, Proc. Natl. Acad. Sci. USA 87: 1874, 1990); Q Beta replicase amplification (see, e.g., Smith, J. Clin. Microbiol. 35: 1477-1491, 1997), automated Q-beta replicase amplification assay (see, e.g., Burg, Mol. Cell. Probes 10: 257-271, 1996) and other RNA polymerase mediated techniques (e.g., NASBA, Cangene, Mississauga, Ontario); see also Berger, Methods Enzymol. 152: 307-316, 1987; Sambrook; Ausubel; U.S. Pat. Nos. 4,683,195 and 4,683,202; Sooknanan, Biotechnology 13: 563-564, 1995.

E. Hybridization of Nucleic Acids

The invention provides isolated or recombinant nucleic acids that hybridize under stringent conditions to an exemplary sequence of the invention, e.g., a Scd1 sequence or toll-like receptor 2 sequence, or the complement of any thereof, or a nucleic acid that encodes a polypeptide of the invention. In alternative aspects, the stringent conditions are highly stringent conditions, medium stringent conditions or low stringent conditions, as known in the art and as described herein. These methods can be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention by its ability to hybridize under stringent conditions to a nucleic acid otherwise determined to be within the scope of the invention (such as the exemplary sequences described herein).

“Stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in e.g., Sambrook, ed., MOLECULAR CLONING: A LABORATORY MANUAL (2ND ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, PART I. Theory and Nucleic Acid Preparation, Tijssen, ed. Elsevier, N.Y., 1993.

Generally, stringent conditions are selected to be about 5-10° C. lower than the thermal melting point I for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide as described in Sambrook (cited below). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

F. Oligonucleotides Probes and Methods for Using Them

The invention also provides nucleic acid probes for identifying nucleic acids encoding a polypeptide which is a modulator of a TLR2- or Scd1-signaling activity. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below. The probes of the invention can also be used to isolate other nucleic acids or polypeptides.

G. Determining the Degree of Sequence Identity

The invention provides nucleic acids having at least 90%, 91%, 92%, 93%, 94%, 95%, 96%, 97%, 98%, 99% or more sequence identity to Scd1 polynucleotide or toll-like receptor 2 polynucleotide. The invention provides polypeptides having at least 90%, 91%, 92%, 93%, 94%, 95%, 96%, 97%, 98%, 99% or more sequence identity to Scd1 protein or toll-like receptor 2 protein. The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

A preferred example of an algorithm that is suitable for determining percent sequence identity and sequence similarity is the FASTA algorithm, which is described in Pearson & Lipman, Proc. Natl. Acad. Sci. U.S.A. 85: 2444, 1988. See also Pearson, Methods Enzymol. 266: 227-258, 1996. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another preferred example of algorithm that is suitable for determining percent sequence identity and sequence similarity are the BLAST and BLAST 2.0 algorithms, which are described in Altschul et al., Nuc. Acids Res. 25: 3389-3402, 1977; and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90: 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35: 351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12: 387-395, 1984.

Another preferred example of an algorithm that is suitable for multiple DNA and amino acid sequence alignments is the CLUSTALW program (Thompson et al., Nucl. Acids. Res. 22: 4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89: 10915-10919, 1992).

“Sequence identity” refers to a measure of similarity between amino acid or nucleotide sequences, and can be measured using methods known in the art, such as those described below:

“Identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

“Homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence of Scd1 gene product or toll-like receptor 2 polynucleotide or polypeptide) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, continugous residues ranging anywhere from 20 to the full length of an exemplary polypeptide or nucleic acid sequence of the invention, e.g., Scd1 or toll-like receptor 2 polynucleotide or polypeptide, are compared to a reference sequence of the same number of contiguous positions after the two sequences are optimally aligned. If the reference sequence has the requisite sequence identity to an exemplary polypeptide or nucleic acid sequence of the invention, e.g., at least 90%, 91%, 92%, 93%, 94%, 95%, 96%, 97%, 98%, 99% or more sequence identity to Scd1 or toll-like receptor 2 polynucleotide or polypeptide, that sequence is within the scope of the invention.

Motifs which can be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

H. Computer Systems and Computer Program Products

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media can be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

### Modulating or Inhibiting Expression of Polypeptides and Transcripts

The Invention Further Provides for Nucleic Acids Complementary to (e.g., antisense sequences to) the nucleic acid sequences of the invention. Antisense sequences are capable of modulating or inhibiting the transport, splicing or transcription of protein-encoding genes, e.g., TLR2- or Scd1-encoding nucleic acids. The modulation or inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides which cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

A. Antisense Oligonucleotides

The invention provides antisense oligonucleotides capable of binding TLR2 or Scd1 messenger RNA which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

B. siRNA

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more. Preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism. Elbashir et al., Genes Dev., 15: 188-200, 2001. First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

C. Inhibitory Ribozymes

The invention provides ribozymes capable of binding message which can inhibit polypeptide activity by targeting mRNA, e.g., inhibition of polypeptides with TLR2 activity or Scd1 activity, e.g., TLR2-signaling activity. Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

### Methods of Treatment

Also described herein are both prophylactic and therapeutic methods of treating a subject at risk of (or susceptible to) a disorder or having a disorder associated with undesirable toll-like receptor 2 expression or activity, or Scd1 gene expression activity or Scd1 gene product activity.

### Prophylactic Methods

The invention relates to methods for preventing in a subject a disease or condition associated with an undesirable amount of toll-like receptor 2 expression or activity, Scd1 gene expression or Scd1 gene product activity, by administering to the subject an agent that modulates signaling through toll-like receptor 2, Scd1 gene expression activity, or Scd1 gene product activity. Subjects at risk for a disorder or undesirable symptoms that are caused or contributed to by toll-like receptor 2- or Scd1-mediated signaling can be identified by, for example, any of a combination of diagnostic or prognostic assays as described herein or are known in the art. In general, such disorders involve undesirable activation of the innate immune system, e.g., as a result of Gram positive bacterial infection. Administration of the agent as a prophylactic agent can occur prior to the manifestation of symptoms, such that the symptoms are prevented, delayed, or diminished compared to symptoms in the absence of the agent. In some embodiments, the agent decreases binding of toll-like receptor 2 to Scd1. In some embodiments, the agent decreases ligand binding to toll-like receptor 2 to Scd1. The appropriate agent can be identified based on screening assays described herein. In general, such agents specifically bind to toll-like receptor 2 and/or Scd1 gene product.

### Therapeutic Methods

Another aspect of the invention pertains to methods of modulating or activating TLR2 activity or Scd1 gene expression or Scd1 gene product activity for therapeutic purposes. The method can include contacting a cell with an agent that modulates one or more of the activities of toll-like receptor 2 and/or Scd1 activity associated with the cell, e.g., specifically binds to TLR2 or Scd1 or activates signaling through toll-like receptor 2. The agent can be a compound that specifically binds to toll-like receptor 2, Scd1 gene, or Scd1 gene product and selectively activates TLR2 activity in a cell that has been induced by lipopolysaccharide, or activates macrophage response to gram positive bacteria. The agent can be an antibody or a protein, a naturally-occurring cognate ligand of a toll-like receptor 2 protein, a peptide, a toll-like receptor 2 or Scd1 protein peptidomimetic, a small non-nucleic acid organic molecule, or a small inorganic molecule. These modulatory methods can be performed in vitro (e.g., by culturing the cell with the agent) or, alternatively, in vivo (e.g., by administering the agent to a subject).

The present invention provides methods for treating an individual affected by a disease or disorder, e.g., Gram positive bacterial infection or Gram positive bacterial skin infection, characterized by lack of expression or activity of a toll-like receptor 2 protein activity, Scd1 gene expression, or Scd1 gene product activity. In one embodiment, the method involves administering a therapeutic agent such as a monounsaturated fatty acid, for example, palmitoleate (palmitoleic acid) or oleate (oleic acid).

The present invention provides methods for treating an individual affected by a disease or disorder characterized by lack of expression or activity of a toll-like receptor 2 protein activity, Scd1 gene expression, or Scd1 gene product activity. In one embodiment, the method involves administering an agent (e.g., an agent identified by a screening assay described herein), or combination of agents that increases signaling through toll-like receptor 2 or increases Scd1 gene expression or Scd1 gene product activity. Conditions that can be treated by agents include those in which a subject is treated for Gram positive bacterial infection.

Other disorders that can be treated by the new methods and compositions include fungal infections, sepsis, cytomegalovirus infection, tuberculosis, leprosy, bone resorption (e.g., in periodontal disease), arthritis (e.g., associated with Lyme disease), and viral hepatitis. Compounds that activate signaling through toll-like receptor 2 (e.g., by activating Scd1 gene expression or Scd1 gene product activity), are also useful for treating Gram positive bacterial infection.

Successful treatment of disorders related to Gram positive bacterial infection can be brought about by techniques that serve to activate binding to toll-like receptor 2, Scd1 gene expression or Scd1 gene product. For example, compounds, e.g., an agent identified using an assay described herein, such as an antibody, that prove to exhibit negative modulatory activity, can be used to prevent and/or ameliorate symptoms of disorders caused by undesirable Scd1 gene product or toll-like receptor 2 activity. Such molecules can include, but are not limited to peptides, phosphopeptides, small organic or inorganic molecules, or antibodies (including, for example, polyclonal, monoclonal, humanized, anti-idiotypic, chimeric or single chain antibodies, and F_{ab}, F(_{ab}′)₂ and F_{ab} expression library fragments, scFV molecules, and epitope-binding fragments thereof). In particular, antibodies and derivatives thereof (e.g., antigen-binding fragments thereof) that specifically bind to toll-like receptor 2 and can modulate or activate Scd1 activity (Scd1 gene expression or Scd1 gene product) in a cell that has been induced by lipopolysaccharide, or modulate or activate macrophage response to gram positive bacterial infection.

### Kits

The invention provides kits comprising the compositions, e.g., nucleic acids, expression cassettes, vectors, cells, polypeptides (e.g., Scd1 polypeptides or toll-like receptor 2-signal activating polypeptides) and/or antibodies of the invention. The kits also can contain instructional material teaching the methodologies and uses of the invention, as described herein.

### Therapeutic Applications

The compounds and modulators identified by the methods of the present invention can be used in a variety of methods of treatment. Thus, the present invention provides compositions and methods for treating an infectious disease, a Gram positive bacterial infection, a toll-like receptor 2 signaling defect, Scd1 gene mutation or gene expression defect or Scd1 gene product defect.

Exemplary infectious disease, include but are not limited to, Gram positive bacterial skin infections, for example, S. pyogenes or S. aureus. Gram positive cocci S. pyogenes or S. aureus are leading agents of human impetigo, cellulites, and wound infection.

Exemplary infectious disease, include but are not limited to, viral or bacterial diseases. The polypeptide or polynucleotide of the present invention can be used to treat or detect infectious agents. For example, by increasing the immune response, particularly increasing the proliferation and differentiation of B and/or T cells, infectious diseases can be treated. The immune response can be increased by either enhancing an existing immune response, or by initiating a new immune response. Alternatively, the polypeptide or polynucleotide of the present invention can also directly inhibit the infectious agent, without necessarily eliciting an immune response.

Similarly, bacterial or fungal agents that can cause disease or symptoms and that can be treated or detected by a polynucleotide or polypeptide of the present invention include, but not limited to, the following Gram-Negative and Gram-positive bacterial families and fingi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. A polypeptide or polynucleotide of the present invention can be used to treat or detect any of these symptoms or diseases.

Moreover, parasitic agents causing disease or symptoms that can be treated or detected by a polynucleotide or polypeptide of the present invention include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. A polypeptide or polynucleotide of the present invention can be used to treat or detect any of these symptoms or diseases.

Preferably, treatment using a polypeptide or polynucleotide of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

### Formulation and Administration of Pharmaceutical Compositions

The invention provides pharmaceutical compositions comprising nucleic acids, peptides and polypeptides (including Abs) of the invention. As discussed above, the nucleic acids, peptides and polypeptides of the invention can be used to activate expression of an endogenous Scd1 gene or Scd1 polypeptide. Such activation in a cell or a non-human animal can generate a screening modality for identifying compounds to treat or ameliorate an infectious disease or Gram positive bacterial infection. Administration of a pharmaceutical composition of the invention to a subject is used to generate a toleragenic immunological environment in the subject. This can be used to tolerize the subject to an antigen.

The nucleic acids, peptides and polypeptides of the invention can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of nucleic acids, peptides or polypeptides of the invention are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Nucleic acids, peptides or polypeptides of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani, Crit. Rev. Ther. Drug Carrier Syst. 13: 85-184, 1996. For topical, transdermal administration, the agents are formulated into ointments, creams, salves, powders and gels. Transdermal delivery systems can also include, e.g., patches.

The nucleic acids, peptides or polypeptides of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (see, e.g., Putney, Nat. Biotechnol. 16: 153-157, 1998).

For inhalation, the nucleic acids, peptides or polypeptides of the invention can be delivered using any system known in the art, including dry powder aerosols, liquids delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The nucleic acids, peptides or polypeptides of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun, Anesth Analg. 85: 317-323, 1997). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's, See also, Bai, J. Neuroimmunol. 80: 65-75, 1997; Warren, J. Neurol. Sci. 152: 31-38, 1997; Tonegawa, J. Exp. Med. 186: 507-515, 1997.

In one aspect, the pharmaceutical formulations comprising nucleic acids, peptides or polypeptides of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble nucleic acids, peptides or polypeptides of the invention have been attached to the surface of the monolayer or bilayer. For example, peptides can be attached to hydrazide-PEG-(distearoylphosphatidyl)ethanolamine-containing liposomes (see, e.g., Zalipsky, Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides and/or polypeptides of the invention are incorporated within micelles and/or liposomes (see, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art, see, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4,235,871, 4,501,728 and 4,837,028.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models, e.g., of inflammation or disorders involving undesirable inflammation, to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, generally of a labeled agent. Animal models useful in studies, e.g., preclinical protocols, are known in the art, for example, animal models for inflammatory disorders such as those described in Sonderstrup (Springer, Sem. Immunopathol. 25: 35-45, 2003) and Nikula et al., Inhal. Toxicol. 4(12): 123-53, 2000), and those known in the art, e.g., for fungal infection, sepsis, cytomegalovirus infection, tuberculosis, leprosy, viral hepatitis, and infection (e.g., by mycobacteria).

As defined herein, a therapeutically effective amount of protein or polypeptide such as an antibody (i.e., an effective dosage) ranges from about 0.001 to 30 mg/kg body weight, for example, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The protein or polypeptide can be administered one or several times per day or per week for between about 1 to 10 weeks, for example, between 2 to 8 weeks, between about 3 to 7 weeks, or about 4, 5, or 6 weeks. In some instances the dosage can be required over several months or more. The skilled artisan will appreciate that certain factors can influence the dosage and timing required to effectively treat a subject, including, but not limited to the severity of the disease or disorder, previous treatments, the general health and/or age of the subject, and other diseases present. Moreover, treatment of a subject with a therapeutically effective amount of an agent such as a protein or polypeptide (including an antibody) can include a single treatment or, preferably, can include a series of treatments.

For antibodies, the dosage is generally 0.1 mg/kg of body weight (for example, 10 mg/kg to 20 mg/kg). Partially human antibodies and fully human antibodies generally have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology, 14: 193, 1997).

The present invention encompasses agents or compounds that modulate expression or activity of Scd1 gene expression or Scd1 gene product by modulating signaling through toll-like receptor 2. An agent can, for example, be a small chemical molecule. Such small chemical molecules include, but are not limited to, peptides, peptidomimetics (e.g., peptoids), amino acids, amino acid analogs, small non-nucleic acid organic compounds or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small chemical molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small chemical molecule depend upon the potency of the small chemical molecule with respect to the expression or activity to be modulated. When one or more of these small chemical molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody or fragment thereof can be linked, e.g., covalently and/or with a linker to another therapeutic moiety such as a therapeutic agent or a radioactive metal ion, to form a conjugate. Therapeutic agents include, but are not limited to, antibiotics (e.g., dactinomycin (formerly actinomycin), bleomycin, mithramycin, and anthramycin (AMC)).

The conjugates described herein can be used for modifying a given biological response. For example, the moiety bound to the antibody can be a protein or polypeptide possessing a desired biological activity. Such proteins can include, for example, a toxin such as abrin, ricin A, Pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers.

Alternatively, an antibody can be conjugated to a second antibody to form an antibody heteroconjugate as described by Segal in U.S. Pat. No. 4,676,980.

The pharmaceutical compositions can be included in a container, pack, or dispenser together with instructions for administration.

Compounds as described herein can be used for the preparation of a medicament for use in any of the methods of treatment described herein.

The pharmaceutical compositions are generally formulated as sterile, substantially isotonic and in full compliance with all Good Manufacturing Practice (GMP) regulations of the U.S. Food and Drug Administration.

### Treatment Regimens: Pharmacokinetics

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, etc. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

In therapeutic applications, compositions are administered to a patient suffering from autoimmune disease, an infectious disease, an antigen presenting cell defect or a CD4 cell defect in an amount sufficient to at least partially arrest the condition or a disease and/or its complications. For example, in one aspect, a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

The following examples of specific embodiments for carrying out the present invention are offered for illustrative purposes only, and are not intended to limit the scope of the present invention in any way.

### EXEMPLARY EMBODIMENTS

### Example 1

### Flake: A Visible Phenovariant with Associated Immunodeficiency

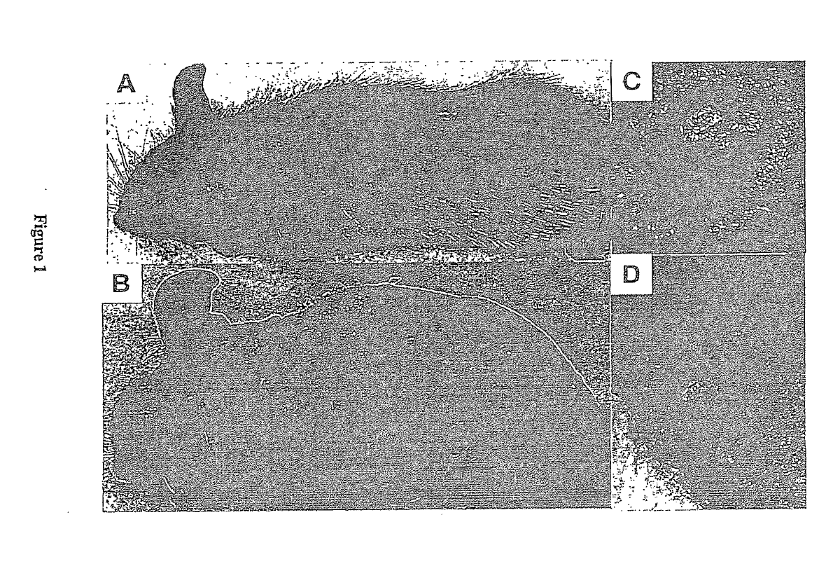

In an effort to identify genes required for normal immune function, a total of 20,792 F1 and 33,202 F3 animals were screened with ENU-induced germline mutations for visible and immunologic phenotypes. Among these, a recessive mutation dubbed “flake” (flk) was found to cause progressive alopecia and chronic exfoliative dermatitis. These features appeared at weaning age and were more pronounced in older animals (FIG. 1). Visible disruption of epidermal integrity and spontaneous skin infections requiring antibiotic therapy prompted us to examine the integrity of innate immune function in these mice.

FIG. 1 shows visible phenotypes observed in flake mutant mice. A. 6-week old mouse. B. 8-month-old mouse. C. Eye infection in an 8-month-old mouse. D. Magnification of the mouse shown in B highlights severe dermatitis

### Example 2

### Persistent Streptococcus pyogenes and Staphylococcus aureus Skin Infections in flk/flk Mutant Mice

The Gram-positive cocci S. pyogenes and S. aureus are the leading agents of human impetigo, cellulitis, and wound infection. Guay, Expert. Opin. Pharmacother. 4:1259-1275, 2003; Hedrick, Paediatr. Drugs 1:35-46, 2003. Experimental full-thickness skin infection in the murine model can be reliably established by immediate subcutaneous injection with a fine-gauge needle, overcoming the requirement for traumatic injury and poor infectivity and reproducibility associated with epicutaneous inoculation. Bunce et al., Infect. Immun. 60:2636-2640, 1992; Kraft et al., Infect. Immun. 52:707-713, 1986; Nizet et al., Nature 414:454-457, 2001.

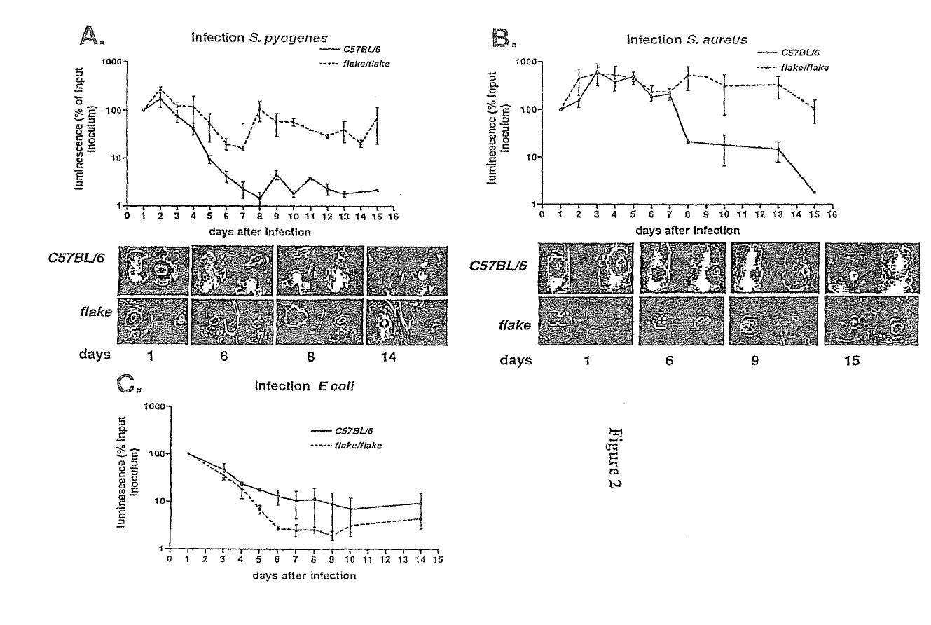

Luminescently-tagged strains of Streptococcus pyogenes, Staphylococcus aureus, and Escherichia coli were utilized, each of which constitutively expressed a bacterial lux operon derived from Photorhabdus luminescens. Kuklin et al., Antimicrob Agents Chemother 47:2740-8, 2003. The progress of each infection was monitored by external luminometry over a period of 16 days in anaesthetized mice. As illustrated in FIG. 2A, normal C57BL/6 mice need 8 days to completely clear a skin infection established by inoculation of 5×10⁵ cfu of S. pyogenes. The flk/flk mutants show similar kinetics of microbial clearance for the first six days following inoculation, but thereafter, the microbial burden in flk/flk mutants departs from control values, rising to reach a plateau that is maintained throughout the duration of the experiment. Luminescence slowly declines to reach background levels 4 weeks after the inoculation in flk/flk mutants.

S. pyogenes produces a small, ulcerated wound, which heals almost completely by day 8 in control mice. Ulceration is still observed in flk/flk mutant mice up to 28 days after infection, albeit without detectable luminescence in vivo. Luminescent S. pyogenes were recovered by culturing the ulcers of flk/flk mutants. Hence, even 4 weeks after experimental inoculation, flk/flk mutant mice remain persistently infected with S. pyogenes.

Infection with S. aureus (FIG. 2B) yields results formally similar to those described above. During the initial period of observation, bacterial burden in flk/flk mutants closely matches that in controls, but a departure in the two curves is observed on day 7 following inoculation, with gradual clearance achieved in control animals (but not in flk/flk mutants), leading to a complete recovery of the controls within 2 weeks. In contrast, luminescence remained strongly detectable in flake mice for more than 3 weeks and reached background levels later than 4 weeks after inoculation.

On the other hand, flk/flk mutants were able to clear an infection with the Gram-negative bacterium Escherichia coli (FIG. 2C). Moreover, no difference between flk/flk mutants and normal controls was observed when Gram-positive infections were introduced by other routes (for example, with intravenous inoculation of L. monocytogenes, or with intrapulmonary challenge using S. aureus). On the basis of all data adduced in these studies, it appears that: 1. flk/flk mutants mice are impaired in their ability to sterilize Gram-positive skin infections; 2. the phenotype does not extend to all biological compartments, and is probably limited to the skin; 3. the single Gram-negative infection examined was not discriminated by the mutation; and 4. the fact that skin lesions induced by E. coli heal normally in flk/flk mice indicates that the mutation does not affect wound healing per se, but rather, has a selective effect on pathogen clearance.

FIG. 2 shows flake mutant mice develop persistent skin infections when exposed to Gram positive bacteria. A. Time-course analysis of the bacterial growth in control (C57BL/6, n=4) and mutant (flake/flake, n=4) animals subcutaneously infected with S. pyogenes. The upper panel shows the graphical representation after luminescence (expressed as a percentage of the initial inoculum) quantification in 4 animals of each genotype. The lower panel shows the overlay of the picture and the light detection for 2 representative mice for each genotype 1, 6, 8 and 14 days after inoculation. B. Infection with S. aureus. Pictures show infected animals at days 1, 6, 9 and 15. C. Infection with E. coli.

### Example 3

### Mapping of the flk Mutation to the Stearoyl CoA Desaturase 1 Locus

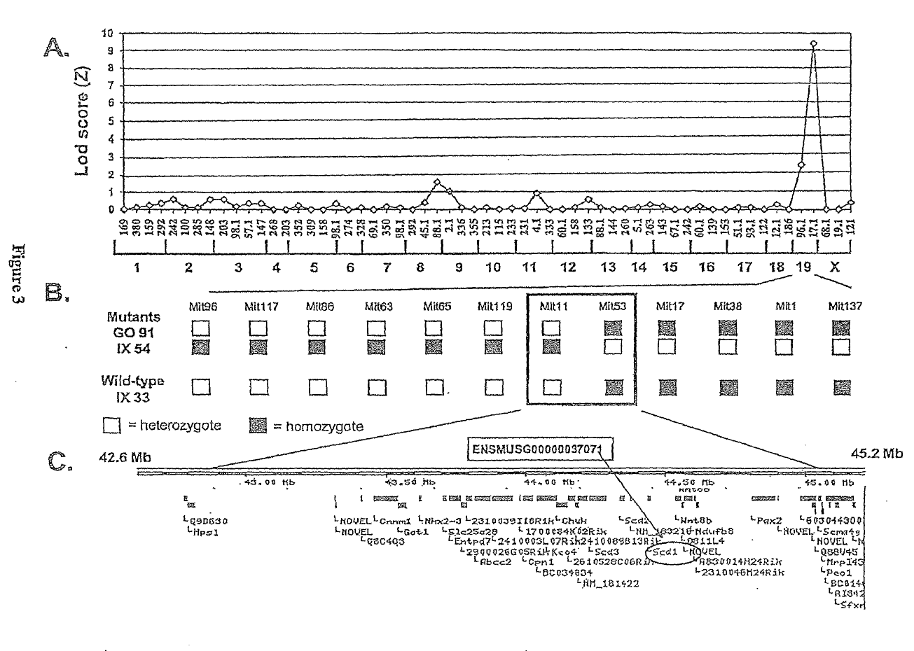

The visible phenotype imparted by flk was utilized in mapping, and concordance between visible and immunologic phenotypes was later established by examining the progeny of intercrossed F1 mice as well as other allelic variants of the locus. flk was initially mapped to chromosome 19 on 39 meioses using a panel of 59 informative markers distributed throughout the mouse genome, in a backcross against C3H/HeN. The phenotype was fully penetrant on the mixed background, and the mutation was placed between markers D19Mit96 and D19Mit17 (FIG. 3A). Fine mapping was then performed using 12 internal chromosome 19 markers, so that on 283 meioses, the mutation was restricted a 2.6 Mbp critical region delimited by D19Mit11 and D19Mit53 (FIG. 3B). Among the 43 genes represented within this region in the Ensembl database (FIG. 3C), the Stearyol CoA desaturase 1 (Scd1) gene was considered as a likely candidate, since two mutant alleles, named asebia-J and asebia-2J, have already been described for Scd1 and in both cases, mutant mice show a cutaneous phenotype described as “scaly skin”, similar to that observed in flk homozygotes. Sundberg et al., Am J Pathol 156:2067-75, 2000; Zheng et al., Nat Genet. 23:268-70, 1999.

FIG. 3 shows mapping of the flake mutation. A. Transgenomic log likelihood ratio (Lod score, Z) analysis shows a single peak of linkage on mouse chromosome 19. A total of 59 informative markers (horizontal axis) were included in the analysis, and 39 meioses (19 wild-type and 17 mutant animals) were genotyped at all markers. B. Fine mapping of the distal region of chromosome 19. Analysis of a total of 283 meioses (3 representative are shown) led to the confinement of the flake mutation between 2 adjacent markers distant by 2.6 Mb. C. Gene organization at the flake locus according to the ENSEMBL database. The Scd1 gene is highlighted.

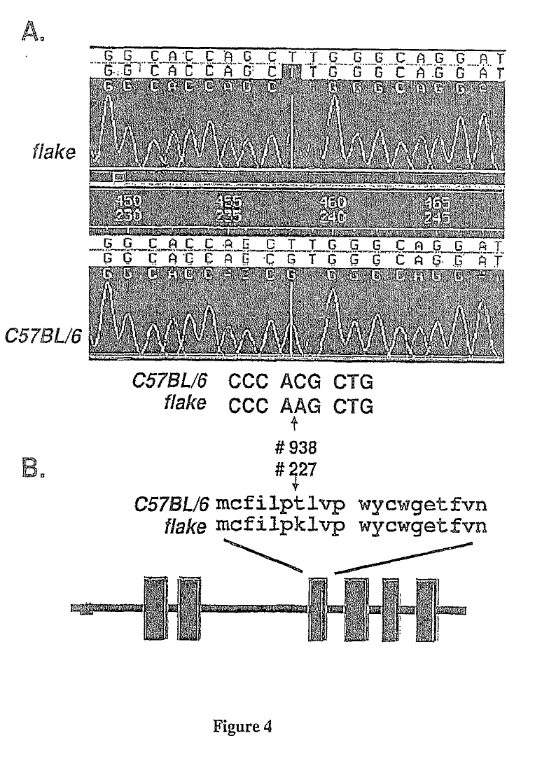

The 6 exons of Scd1 were amplified from genomic DNA isolated from both C57BL/6 control mice and flk/flk mutants. Direct sequencing of the amplicons revealed a point mutation (C to A) in exon 5, which corresponds to position #938 in the cDNA sequence (Accession Number BC055453, see FIG. 4A). This ENU-induced base transversion is predicted to cause a missense mutation (T227K) within SCD1. No mutation was detected in Scd2 and Scd3 cDNAs.

The microsomal enzyme SCD1 is an iron-binding 41 kDa protein of 355 amino acids with six predicted transmembrane domains. It catalyses Δ9-desaturation of long-chain unsaturated fatty acids, leading to the biosynthesis of palmitoleate (C16:1) and oleate (C18:1) as its major products. As illustrated in FIG. 4B, the substitution of a neutral amino acid (T) for a charged residue (K) in the mutated protein occurs within a predicted transmembrane domain, and would be expected to disrupt the structural integrity of SCD1.

FIG. 4 shows molecular characterization of the flake mutation. A. Trace file of amplified genomic DNA from homozygous flake mutant mice (top chromatogram) and normal animals (bottom chromatogram). B. Schematic representation of the SCD1 protein and localization of the flake mutation. Blue boxes correspond to transmembrane domains predicted by SMART analysis.

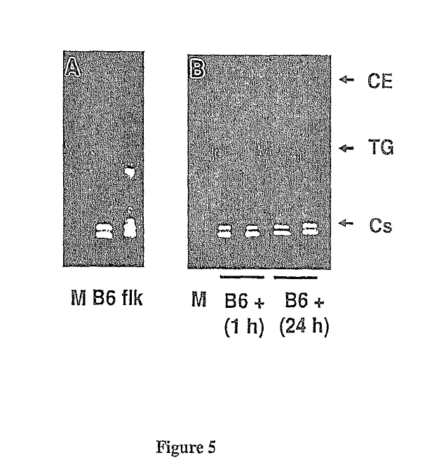

To test this assumption, thin layer chromatography (TLC) was performed to analyze the lipid composition of skin biopsies from control and flk/flk mice. The latter animals exhibit a reduction in cholesterol esters (FIG. 5A), similar to that reported in the case of Scd1 KO, which indicates that the flk phenotype is indeed caused by the observed allelic variant of Scd1.

FIG. 5 shows thin layer chromatography analysis of the lipid contend in wild-type and flake mutant mice. A. TLC of lipids extracted from skin biopsies of wild-type (B6) or flake (flk) mutant mice. B. TLC of lipids purified from the skin of wild-type mice (B6+) 1 hour or 24 hours after S. aureus subcutaneous infection. M: Markers. Cs: Cholesterol, TG: Triglycerides, CE: Cholesterol Esters.

### Example 4

### Palmitoleate and Oleate have Intrinsic Antibacterial Activity In Vitro and In Vivo

The absence of C18 and C16 fatty acid desaturase activity in Scd1^{flk/flk} mutant mice prompted us to ask whether the lack of oleate and/or palmitoleate could account for the cutaneous immunodeficiency phenotype described above. Indeed, several reports have indicated that MUFA exhibit antimicrobial activity against Gram-positive bacteria, though there is no evidence that MUFA exert a protective effect in vivo. Miller et al., Arch Dermatol 124:209-15, 1988; Wille and Kydonieus, Skin Pharmacol Appl Skin Physiol 16:176-87, 2003. To test the working hypothesis, a series of in vitro experiments were first performed in which the effect of each lipid was measured on the growth of S. pyogenes, S. aureus and E. coli.

The results confirmed that both palmitoleate and oleate each have strong bacteriostatic and bactericidal activity against S. pyogenes and S. aureus. The minimum inhibitory concentration (MIC, see Table 1) of both compounds on S. pyogenes is in the micromolar range, and comparable to that observed for the murine cathelicidin AMP (CRAMP). On a weight basis, the MUFA are therefore approximately 20 times as potent as cathelicidin. MUFA are also active against S. aureus, whereas CRAMP is totally inactive. On the other hand, no bacteriostatic or bactericidal activity was detected against E. coli even at millimolar MUFA concentrations, consistent with a specific effect against Gram-positive bacteria.

TABLE 1
Minimum Inhibitory Concentration (MIC) and Minimum Bactericidal Concentration
(MBC), expressed in μM of cathelicidin antimicrobial peptide (CRAMP),
oleic acid and palmitoleic acid on S. pyogenes and S. aureus.

| | MIC (μM) | | | MBC (μM) | | |
|---|---|---|---|---|---|---|
| | CRAMP | Oleate | Palmitoleate | CRAMP | Oleate | Palmitoleate |
| S. pyogenes | 5.3 +/− 2.3 | 8.3 +/− 2.9 | 10 +/− 0.1 | 11.3 +/− 5.8 | 13.3 +/− 5.8 | 10 +/− 0.1 |
| S. aureus | Resistant | >75 | 36.6 +/− 11.5 | Resistant | nd | 50 +/− 15 |

Values represent the average of 3 experiments. nd, not determined.

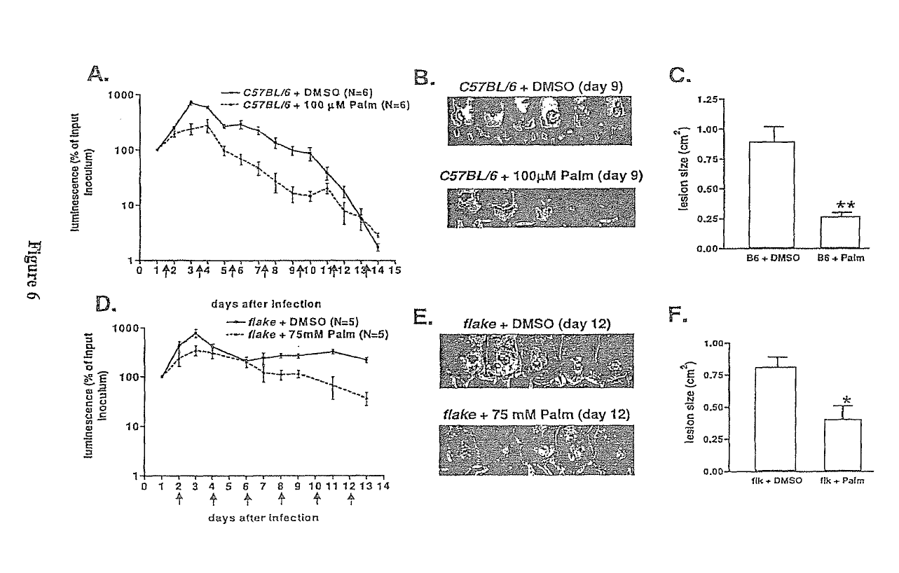

To investigate the physiological relevance of this antimicrobial activity, wild-type mice were inoculated with S. aureus and treated the infected animals by repeated (every two days) subcutaneous injections of palmitoleate (100 μl of a 100 μM solution in DMSO), or DMSO alone at the site of infection. The results of this experiment are illustrated in FIGS. 6A and B. For both groups of mice (n=6 animals), luminescence is expressed as a percentage of the initial inoculum, determined 24 hours after infection. 9 days after S. aureus inoculation, palmitoleate-treated animals exhibit a 90% reduction of luminescence, compared to vehicle-treated mice. As a consequence of improved S. aureus elimination, the diameter of the ulcerative wound (measured at day 9) in lipid-treated animals is one fourth that observed in controls (FIG. 6C). These data, which clearly illustrate the antibacterial capacities of MUFA in vitro and in vivo, also reveal that this lipid-based defense mechanism is not maximally efficacious in normal mice.

Under similar conditions of palmitoleate administration, flake mutants exhibited a marked reduction of bacterial growth between days 1 and 4 (also observed in wild-type mice), but S. aureus remained detectable 2 weeks after inoculation. As illustrated FIGS. 6D and E, a higher dose of palmitoleate (100 μl of a 75 mM solution) moderately improves bacterial clearance in flk/flk mutants and the subsequent ulcer healing (FIG. 6F). However, complete rescue of the phenotype was not achieved by this pharmacological approach.

FIG. 6 shows palmitoleic acid has antibacterial activity in vivo. A. Palmitoleate injection accelerates bacterial clearance in wild-type mice. Luminescence (expressed as a percentage of the initial inoculum) was measured in control (C57BL/6) mice inoculated with S. aureus (at day 0) and treated by vehicle (DMSO) or palmitoleate injections every two days (arrows). B. Picture of control (C57BL/6) mice 9 days after S. aureus infection treated by DMSO (top) or palmitoleate (bottom) injections. C. Histogram showing the size of the lesion measured at day 9 after the infection in control (B6) mice treated with DMSO or palmitoleate. ** indicates P value <0.01. D. Palmitoleate treatment in S. aureus-infected flake mice. The protocol is similar as in A, except that 100 μl injections of a 75 mM solution of palmitoleate were performed. E. Pictures of infected flake mice at day 12 after DMSO (top) or palmitoleate (bottom) treatment. F. Size of the lesion (determined at day 12) in infected flk mutants treated with DMSO or palmitoleate. * indicates P value <0.05.

### Example 5

### Transcriptional Activation of Scd1 Occurs During Gram-Positive Bacterial Infection and is TLR2-Dependent

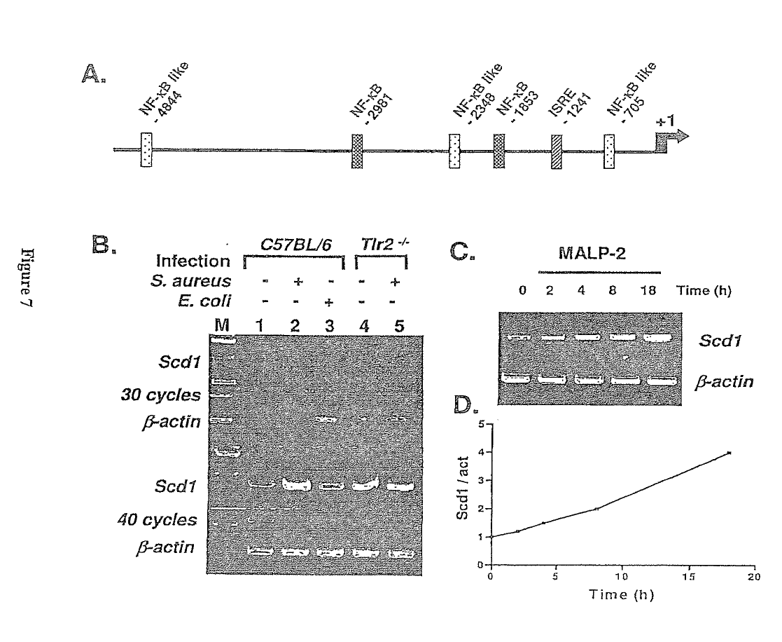

The unsuspected in vivo antimicrobial function of MUFA prompted us to ask whether their synthesis is increased during the immune response, as is the case for other effector molecules such as CRAMP. a 5 kb fragment of the Scd1 promoter were analyzed and the presence of several NF-κB binding sites was noted (FIG. 7A). semi-quantitative RT-PCR experiments was performed on skin biopsies from normal or infected mice. FIG. 7B illustrates that Scd1 mRNA accumulation is strongly induced in the skin of control (C57BL/6) mice upon S. aureus infection, whereas E. coli inoculation produces no effect. Furthermore, in mice carrying a targeted disruption of the Tlr2 gene (Tlr2^{-/-}) the Scd1 gene is unresponsive to inoculation of Gram-positive bacteria. However, Scd1 transcriptional induction might also be caused by an indirect mechanism, given the 24 hour delay between infection and RNA isolation.

FIG. 7 shows infection- and TLR2-dependant induction of Scd1 gene expression in mice. A. SignalScan analysis of the Scd1 promoter. NF-κB and ISRE (interferon-stimulated regulatory element) are shown. B. RT-PCR detection of Scd1 and β-actin transcripts in skin biopsies of non-infected controls (C57BL/6, lane 1) and Tlr2−/− (lane 4) animals or after infection by S. aureus (lanes 2 and 5) or E. coli (lane 3). PCR products after 30 and 40 cycles are shown. M, size standard. C. RT-PCR detection of Scd1 and β-actin transcripts in controls (0) and MALP-induced peritoneal macrophages isolated from wild-type mice after 2, 4, 8 and 18 h. D. Quantification of the Scd1/β-actin ratio.

Macrophages, which represent an ideal system in which to study TLR signaling, also express the Scd1 gene, as reported recently. Uryu et al., Biochem Biophys Res Commun 303:302-5, 2003. To determine whether isolated macrophages are capable of upregulating Scd1 and to determine the kinetics of the response, peritoneal macrophages isolated from wild-type mice were stimulated with, synthetic macrophage-activating lipopeptide (MALP-2, EMC microcollections GmbH, Germany), a known TLR2 agonist. Takeuchi et al., J Immunol 164:554-7, 2000. Scd1 expression was surveyed by RT-PCR on RNA samples isolated 2, 4, 8 and 18 hours after stimulation. As seen on FIGS. 7C and D, Scd1 expression is augmented 2 h after MALP induction and reaches a 4-fold increase within 18 hours. This transcriptional induction of Scd1 was correlated to an increased lipid synthesis in the skin of infected animals (see FIG. 5B).

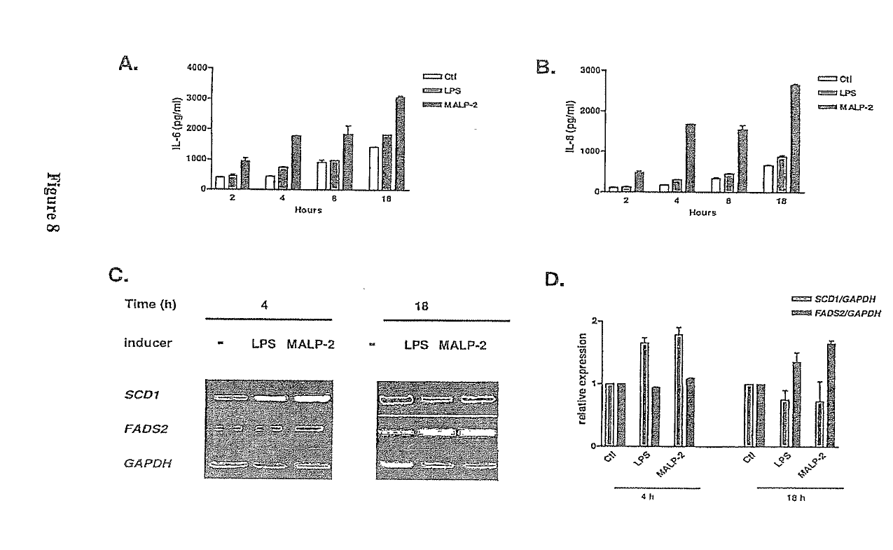

As previously noted, Scd1 is expressed principally in sebaceous glands and flake, as well as asebia and Scd1 KO mice, exhibit atrophy of these structures. To corroborate potential relevance of inducible Scd1 expression in human skin defense against Gram-positive pathogens, the effect of MALP-2 was investigated on the immortalized human sebocyte cell line SZ95. Zouboulis et al., J. Invest Dermatol. 113:1011-1020, 1999. First, MALP-2, but not LPS treatment, induced a rapid and potent inflammatory response, manifested by increased IL-6 and IL-8 production (FIGS. 8A and B). Next, it was observed that SCD1 transcription is also up-regulated in this human cell line 4 hours after MALP-2 stimulation (FIGS. 8C and D). These observations were extended by monitoring the expression of the fatty acid desaturase2 (FADS2) gene. FADS2 encodes a protein with enzymatic properties similar to those of SCD1 and was recently shown to be deficient in a patient affected by a severe skin condition manifested by cheilosis, a hyperkeratotic rash over the arms and legs and perineal dermatitis. Williard et al., J. Lipid Res. 42:501-508, 2001. In human sebocytes, FADS2 is slightly but specifically induced 18 hours after MALP-2 stimulation.

FIG. 8 shows human sebocytes stimulated with MALP-2 show an inflammatory response and up-regulation of SCD1 and FADS2 genes. A. IL-6 production is induced in SZ95 cells after MALP-2 treatment (50 ng/ml). LPS stimulation (100 ng/ml) shows minimal effect. B. Quantification of IL-8 in the same conditions as in A. C. RT-PCR detection of SCD1 and FADS2 expression 4 and 18 hours after LPS and MALP-2 stimulation. GAPDH expression was used as control. D. Quantification of the SCD1 and FADS2 signals measured in two independent experiments (+/− s.e.m) after normalization with the GAPDH signal.

### Example 6

### A Toll-Like Receptor 2-Responsive Lipid Effector Pathway Protects Mammals Against Gram-Positive Bacterial Skin Infections

SCD1 is an enzyme responsible for the biosynthesis of MUFA, mainly palmitoleate (C16:1) and oleate (C18:1). Ntambi, Prog Lipid Res 34:139-50, 1995. It catalyses Δ9 cis desaturation of the carbon chain, and uses palmitoyl-CoA and stearoyl-CoA as substrates. The functions of this enzyme in lipid metabolism have been intensely studied. Ntambi and Miyazaki, Prog Lipid Res 43:91-104, 2004. Scd1^{-/-} mice are significantly leaner than wild-type animals and are resistant to diet-induced adiposity, an effect mediated by increased expression of genes involved in fatty acids oxidation. Furthermore, compound homozygotes for hypomorphic mutations of the obese (ob) and Scd1 genes exhibit a striking attenuation of the obese phenotype. Ntambi et al., Proc Natl Acad Sci 99:11482-6, 2002. The observation that Scd1 is overexpressed in ob mutants indicates that at least part of the leptin's metabolic actions results from the inhibition of Scd1. Cohen et al., Science 297:240-3, 2002. Two spontaneous mutant alleles of Scd1 have been described and named asebia (ab) −J and −2J. Sundberg et al., Am J Pathol 156:2067-75, 2000; Zheng et al., Nat Genet. 23:268-70, 1999. Despite minor phenotypic differences, homozygosity for each of these alleles is associated with atrophic sebaceous glands, alopecia and scaly skin, phenotypes which are also observed in mice carrying a targeted disruption of the gene. Miyazaki et al., J Nutr 131:2260-8, 2001.

The present study, provides a mutation, flake, a visible recessive phenovariant with a highly selective innate immunodeficiency phenotype, in which there is failure to eliminate Gram-positive (but not Gram-negative) organisms from the skin. Using a phenotype-driven approach, the flk mutation was tracked to a missense error (T227K) that falls within the fourth of six transmembrane domains of the SCD1 protein. The replacement of a neutral by a charged residue in such a region might alternatively modify the conformation of the desaturase, which normally resides within microsomal membranes, or affect coordination of the iron atom that is necessary for enzymatic activity. Whatever the mechanism, a reduction was demonstrated in the level of cholesterol esters (the biosynthesis of which requires MUFA) in lipid isolates from the skin of flake mutant mice, confirming that the new allele is hypofunctional.

Herein, SCD1 and the products of its catalytic activity in epithelial innate immunity against Gram-positive bacteria were implicated. It has previously been shown that feeding Scd1 deficient mice a MUFA-enriched diet does not alleviate the mutant phenotype, which indicates that de novo synthesis of MUFA is required for normal appearance and function of the skin. Therefore, to extend the in vitro observations, the affect of intradermal administration of palmitoleate to S. aureus-infected mice was monitored. These in vivo experiments showed that repeated subcutaneous injections of palmitoleate reduced bacterial proliferation and significantly improved the recovery of infected mice, as evidenced by reduction of the ulcerative wound. However, this beneficial effect of palmitoleic acid was less pronounced in flake mutants, despite repeated injections of higher doses of palmitoleate. The over-activated lipid catabolism observed in Scd1 mutants might lead to a shorter half-life of the injected lipids and could explain this discrepancy. Nevertheless, it was noted that humans treated for acne problems with retinoids (which induce atrophy of the sebaceous glands) can suffer recurrent S. aureus skin infections as a side effect. Leyden et al., J Invest Dermatol 86:390-3, 1986. Gram-positive bacterial infections of the eye have also been noted in such patients. Egger et al., Opthalmologe 92:17-20, 1995. Indeed, eye infections were also observed in flake mutants (see FIG. 1C), as earlier noted for Scd1 KO mice. Miyazaki et al., J Nutr 131:2260-8, 2001. The data from flk/flk mice emphasize the essential role of sebaceous glands, as well as other lipid-producing organs, including perhaps the specialized Meibomian glands of the eyelids, in local innate immune responses.

The mechanism by which MUFA selectively lyse Gram positive bacteria remains to be determined. The length of the carbon chain and/or the level of unsaturation might be important determinants of efficacy. In addition, synergy between lipids and AMP might also be examined. Flake/CRAMP double knock-out mice will prove to be useful tools with which to study this issue. The experiments do not exclude the possibility that, in addition to their antimicrobial activity, palmitoleate and oleate might promote resistance indirectly. Modulation of signal transduction through protein modification might be one such mechanism. As reported, mass spectrometry identified palmitoleate among other post-translational modifications of src homology domain 3 kinase Fyn, which might affect immune cell activation, as recently shown for insulin signaling in muscle cells. Liang et al., J Biol Chem 279:8133-9, 2004; Rahman et al., Proc Natl Acad Sci 100:11110-5, 2003.

SCD1 transcription is strongly upregulated in mouse and human cells in a TLR2-dependent manner. Human patients with rare skin disorders such as the syndrome of ichthyosis follicularis with atrichia and photophobia (IFAP syndrome, OMIM 308205) possess atrophic sebaceous glands, and coincidentally suffer alopecia and recurrent skin infections reminscent of the Flake phenotype (reviewed in Alfadley et al., Pediatr. Dermatol. 20:48-51, 2003). With new recognition that TLR2 and 6 are expressed in human sebocytes (Zouboulis et al., in preparation), the results point to a prominent and unsuspected role of the sebaceous gland in the skin innate immune defense. Altogether, the data demonstrate the existence of an inducible lipid-based microbicidal effector pathway in the skin, and establish a clear functional link between lipid metabolism and innate immunity.

### Example 7

### Materials and Methods

Mice. Germline mutagenesis using N-ethyl-N-nitrosourea (ENU) was described in. Hoebe et al., J Endotoxin Res 9:250-5, 2003. Animals were maintained under pathogen-free conditions in the animal care facility of the Immunology Department of The Scripps Research Institute. All mice used in the experiments were 8-12 weeks in age. Handling of mice and experimental procedures were conducted in accordance with institutional guidelines for animal care and use.

Bacteria. S. aureus Xen8.1 (parental strain 8325-4), S. pyogenes Xen20 (derived from serotype M49, strain 591) and E. coli Xen14 (derived from EPEC WS2572) were obtained from Xenogen (Carnbury, N.J.)

Cell culture. SZ95 sebocytes were maintained in HSG-Med (Sebomed, Berlin, Germany) supplemented with 10% heat inactivated FCS, 5 ng/ml human epidermal growth factor, 1 mM CaCl₂, 10⁻⁵ M palmitic acid, 50 μg/ml gentamicin for 2, 4, 8 and 18 hours with/without 50 ng/ml MALP-2 or 100 ng/ml LPS and the supernatants were collected for IL6 and IL8 evaluation by ELISA. RNA was isolated from the 4- and 18-hour samples by the RNeasy Midi kit (Qiagen, Hilden, Germany) and purified by the RNase-Free DNase set (Qiagen) for RT-PCR.

Reagents. Palmitoleic and oleic acids were purchased from Sigma. S. minesota Re595 LPS was obtained from Alexis (Carlsbad, Calif.) and MALP-2 from EMC microcollections GmbH (Tübingen, Germany).

Skin infection. Bacterial cultures in exponential growth phase were centrifuged and the pellet was resuspended in 10 volumes of PBS containing 10 mg/ml of inert Cytodex beads (Sigma) used as a carrier. Approximately 5×10⁵ c.f.u of luminescent bacteria in 100 μl were injected subcutaneously on the back of anesthetized animals. Hairs were removed by chemical depilation prior to inoculation. Luminescence was monitored daily with a CCD camera (5 min exposure of the animals) and quantification was done with the IVIS program from Xenogen.

Thin layer chromatography. Total lipids extracted from skin biopsies by chloroform/methanol were separated by silica gel TLC. Hexane/diethyl ether/Acetic acid (70:30:1) was used as developing solvent and lipids were visualized under a UV lamp after spaying a primuline solution (5 mg in 100 ml acetone/water, 80/20).

Semi-quantitative RT-PCR. Wild-type and Tlr2^{-/-} mutant mice were depilated and infected by subcutaneous injection of S. aureus or E. coli (5×10⁵ pfu). After 24 h, the skin of the infected area was dissected and total RNA was extracted by the Trizol (Gibco) method. 1 μg of RNA was used to synthesize oligodT-primed cDNA (Retroscript™, Ambion) which then served as template in PCR reactions using primers specific for Scd1 (3′-ctctatggatatcgcccctacgacaagaacattc-5′ in exon 5 and 3′-gaagctaggaacaaggagggatgtattcaggagg-5′ in exon 6 which allow distinction between genomic and cDNA amplification) or β-actin genes. 4 μl of the PCR reactions were loaded on agarose gels. Isolation of peritoneal macrophages and stimulation has been described elsewhere. Hoebe et al., J Endotoxin Res 9:250-5, 2003. hSCD1 and hFADS2 expression SZ95 sebocytes was measured by semi-quantitative RT-PCR using the following oligonucleotides:

hSCD1f 5′-TTCAGAAACACATGCTGATCCTCATAATTCCC-3′,
hSCD1r 5′-ATTAAGCACCACAGCATATCGCAAGAAAGTGG-3′
hFADS2f 5′-ACTTTGGCAATGGCTGGATTCCTACCCTC-3′
hFADS2r 5′-ACATCGGGATCCTTGTGGAAGATGTTAGG-3′

Glyceraldehyde-3-phosphate dehydrogenase (GAPDH) expression was used as control.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method for treating Gram positive bacterial infection in a mammalian subject comprising administering to the subject an effective amount of a compound that activates Scd1 gene expression or gene product.

2. The method of claim 1 wherein the compound is an agonist of toll-like receptor 2.

3. The method of claim 1 wherein the compound is a small chemical molecule, an antibody, an antisense nucleic acid, short hairpin RNA, or short interfering RNA.

4. The method of claim 1 wherein the Gram positive bacterial infection is Streptococcus pyogenes infection or Staphlococcus aureus infection.

5. The method of claim 2 wherein the subject has a loss-of-function or reduced function mutation in the Scd1 gene.

6-15. (canceled)

16. A method for treating Gram positive bacterial infection in a mammalian subject comprising administering to the subject an effective amount of a compound that is a product of the Scd1 biosynthetic pathway.

17. The method of claim 16 wherein the compound is a monounsaturated fatty acid.

18. The method of claim 17 wherein the monounsaturated fatty acid is palmitoleate or oleate.

19. The method of claim 16 wherein the Gram positive bacterial infection is Streptococcus pyogenes infection or Staphlococcus aureus infection.

20. The method of claim 16 wherein administration of the effective amount of the monounsaturated fatty acid is topical or intradermal.

21. The method of claim 16 wherein administration of the effective amount of the monounsaturated fatty acid is intramuscular, subcutaneous, intraperitoneal, or intravenous.

22. A method for identifying a compound which modulates Gram positive bactericidal activity in cells comprising:
contacting the test compound with a cell-based assay system comprising a cell expressing toll-like receptor 2,
providing a ligand to the assay system in an amount selected to be effective to activate toll-like receptor 2 signaling, wherein toll-like receptor 2 signaling is capable of signaling responsiveness to the ligand and modulating Scd1 gene expression, and
detecting an effect of the test compound on toll-like receptor 2 signaling and on modulation of Scd1 gene expression, effectiveness of the test compound in the assay being indicative of the Gram positive bacteriocidal activity.

23. The method of claim 22 wherein the ligand is an endogenous ligand or an exogenous ligand.

24. The method of claim 23 wherein the exogenous ligand is lipopolysaccharide, lipid A, di-acylated lipopeptide, tri-acylated lipopeptide, S-MALP-2, R-MALP-2, bacterial lipopeptide, Pam2CSK4, lipoteichoic acid, or zymosan A.

25. The method of claim 24 wherein the exogenous ligand is S-MALP-2 or R-MALP-2.

26. The method of claim 23 wherein the exogenous ligand is rough lipopolysaccharide, smooth lipopolysaccharide, or lipid A from Salmonella minnesota.

27. The method of claim 23 wherein the detecting step further comprises measuring activation of Scd1 gene expression or Scd1 gene product in the cell, wherein Scd1 gene expression or Scd1 gene product is activated in response to contacting the cell with the exogenous ligand.

28. The method of claim 27 wherein the exogenous ligand is a component Gram positive bacteria and not a component of Gram negative bacteria.

29. The method of claim 23 wherein the endogenous ligand is a lipid.

30-45. (canceled)

46. A method for diagnosing a risk factor for Gram positive bacterial infection in a mammalian subject comprising:
removing cells or tissue from the subject,
contacting the cells or tissue with an endogenous ligand or exogenous ligand to toll-like receptor 2,
measuring production of Scd1 gene product in the cells or tissue contacted by the ligand, and
detecting reduced function or loss of function of the Scd1 gene product in the mammalian subject.

47. The method of claim 46 wherein the cells or tissue are from macrophage, sebocyte, or sebaceous gland.

48-52. (canceled)

53. The method of claim 46 wherein the ligand is an exogenous ligand, lipotechoic acid (LTA), di-acylated lipopeptide, tri-acylated lipopeptide, S-MALP-2, bacterial lipopeptides, peptidoglycan, mannans, unmethylated CpG DNA, flagellin, or single-stranded RNA.

54. The method of claim 46 wherein the exogenous ligand is S-MALP-2.

55. The method of claim 46 wherein the ligand is an endogenous ligand, lipid, fat, sterol, lipoprotein, fatty acid, oxidized LDL, thrombospondin, or β-amyloid.

56-68. (canceled)
